(19) United States
(12) Patent Application Publication
(10) Pub. No.: US 2026/0299144 A1
(43) Pub. Date: Oct. 1, 2026

(54) METHOD FOR REDUCING OPTICAL CROSSTALK IN SIPM RADIATION DETECTORS

(71) Applicant: Cintilight, LLC, Seattle, WA (US)

(72) Inventors: Philipp Braeuninger-Weimer, Seattle, WA (US); Chad E. Seaver, Knoxville, TN (US)

(73) Assignee: Cintilight, LLC, Seattle, WA (US)

(21) Appl. No.: 19/403,248

(22) Filed: Nov. 28, 2025

Related U.S. Application Data
(60) Provisional application No. 63/777,670, filed on Mar. 25, 2025.

(51) Int. Cl. G01T 1/20; A61B 6/03; G01T 1/202

(52) U.S. Cl. CPC G01T 1/20183; A61B 6/037; G01T 1/202

(57) ABSTRACT

Radiation detection systems and modules include scintillation crystals with silicon photomultiplier (SiPM) arrays on at least one face. The SiPM or SiPMs are operated in an overvoltage state to enhance detection sensitivity, which increases unwanted optical crosstalk. Various examples of spectrally selective crosstalk rejectors, such as layers, materials, films, and the like, are configured to transmissively pass higher wavelength scintillation photons while rejecting or absorbing lower-wavelength external crosstalk photons generated by neighboring SiPMs.

### PRIORITY APPLICATIONS

This application claims priority to and benefit under 35 U.S.C. § 119 to U.S. Provisional Patent Application No. 63/777,670, filed on Mar. 25, 2025, entitled “Method for Reducing Optical Crosstalk in High-Bias SiPM Radiation Detectors,” which is hereby incorporated by reference in its entirety.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc., applications of such applications are also incorporated by reference, including any priority claims made in those applications and any material incorporated by reference, to the extent such subject matter is not inconsistent herewith.

### CROSS REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 U.S.C. § 119(e) for provisional patent applications, for any and all parent, grandparent, great-grandparent, etc., applications of the Priority Application(s)). In addition, the present application is related to the “Related Applications,” if any, listed below.

### RELATED APPLICATIONS AND PUBLICATIONS

This application is also related to and can be understood in the context of U.S. Pat. No. 12,449,554, granted on Oct. 21, 2025, entitled “Scintillator Detectors and Methods for Positron Emission Tomography,” which is hereby incorporated by reference in its entirety.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc., applications of the Priority Applications and the Related Applications, including any priority claims, is incorporated herein by reference to the extent such subject matter is not inconsistent herewith.

### TECHNICAL FIELD

The present disclosure relates to radiation detection and imaging. More specifically, this disclosure relates to crosstalk suppression in semiconductor optoelectronic devices that employ scintillators and silicon photomultipliers (SiPMs).

### BRIEF DESCRIPTION OF THE DRAWINGS

FIG. 1A illustrates a perspective view of a block diagram of a portion of a positron emission tomography (PET) detector system, according to one embodiment.

FIG. 1B illustrates another view of a block diagram of a portion of the PET detector system, according to one embodiment.

FIG. 2 illustrates a positron source with multiple lines of response, according to one embodiment.

FIG. 3 shows a tube-based block detector with four photomultiplier tubes, according to one embodiment.

FIG. 4 is a lock diagram of a ring of detector modules with multiple lines-of-response, according to one embodiment.

FIG. 5A shows a coordinate system used with respect to a scintillator detector geometry, according to one embodiment.

FIG. 5B illustrates simplified block diagrams of various scintillator detector readout configurations, according to various embodiments.

FIG. 5C illustrates a graph of the effective sensitivity of lateral side readout configurations for various thicknesses of lateral detectors, according to one embodiment.

FIG. 6A illustrates the sensor-plane light spread with a thick interface layer between the silicon photomultipliers (SiPMs) and the scintillation crystal, according to one embodiment.

FIG. 6B illustrates the sensor-plane light spread with a thin interface layer between the SiPMs and the scintillation crystal, according to one embodiment.

FIG. 6C illustrates the sensor-plane light spread with a thin interface layer between the SiPMs and an array of pixelated scintillation crystals, according to one embodiment.

FIG. 6D illustrates the sensor-plane light spread with a thick interface layer between the SiPMs and an array of pixelated scintillation crystals, according to one embodiment.

FIG. 7A illustrates high-aspect-ratio pixelated scintillation crystals in a 5×5 array coupled to a 4×4 SiPM array, according to one embodiment.

FIG. 7B illustrates a monolithic scintillator with a 5×5 array of SiPMs in a one-sided end-readout configuration, according to one embodiment.

FIG. 7C illustrates a monolithic scintillator with a two-sided readout configuration of SiPM arrays on an end-face and one lateral face, according to one embodiment.

FIG. 7D illustrates a monolithic scintillator with a three-sided readout configuration of SiPM arrays on an end-face and two lateral faces, according to one embodiment.

FIG. 7E illustrates a scintillation event within the monolithic scintillation crystal with the three-sided readout configuration of FIG. 7D, according to one embodiment.

FIGS. 8A-8I illustrates various configurations of spectrally selective crosstalk rejectors used in conjunction with scintillator detectors with SiPMs coupled to scintillation crystals, according to various embodiments.

FIG. 9 illustrates a graph of lutetium-yttrium oxyorthosilicate (LYSO) emission wavelengths and the product of SiPM photodetection efficiency (PDE) and the SiPM external crosstalk emission spectra, according to one embodiment.

FIG. 10 illustrates a graph of a target absorption spectrum for a spectrally selective crosstalk rejector, according to one embodiment.

FIG. 11 illustrates examples of narrow-band absorption profiles for several optical dyes suitable for use in spectrally selective crosstalk rejectors, according to various embodiments.

FIG. 12 illustrates a rejection spectrum for an example spectrally selective crosstalk rejection material, according to one embodiment.

FIG. 13 illustrates graphs of detected dark count rate (DCR) for an SiPM as a function of overvoltage with and without a rejection layer, according to one embodiment.

FIG. 14 illustrates a graph of the number of detected events as a function of overvoltage for an example scintillator detector with SiPMs on three orthogonal sides, according to one embodiment.

FIG. 15A illustrates a graph of the coincident time resolution (CTR) with respect to overvoltage values for an example scintillator detector with and without a spectrally selective crosstalk rejector (“rejection layer”), according to one embodiment.

FIG. 15B illustrates a graph of the measured photopeak energy as a function of overvoltage for the example scintillator detector with and without the rejection layer, according to one embodiment.

FIG. 15C illustrates a graph of the energy resolution as a function of overvoltage for the example scintillator detector with and without the rejection layer, according to one embodiment.

FIG. 15D illustrates a graph of the number of coincidence events as a function of overvoltage for the example scintillator detector with and without the rejection layer, according to one embodiment.

FIG. 16 illustrates a graph of the acquisition time as a function of overvoltage for the example scintillator detector with and without the rejection layer, according to one embodiment.

FIG. 17 illustrates a graph of the leakage current as a function of overvoltage for the example scintillator detector with and without the rejection layer, according to one embodiment.

FIG. 18A illustrates an example of a radiation detector module with four scintillator detectors arranged in a 2×2 array, according to one embodiment.

FIG. 18B illustrates a radial sector with four radiation detector modules, according to one embodiment.

FIG. 19A illustrates an example of a radiation detector module with four scintillator detectors arranged in a 2×2 array with spectrally selective crosstalk rejection layers between the SiPMs and the faces of the scintillation crystal, according to one embodiment.

FIG. 19B illustrates the example radiation detector module of FIG. 19A with one of the scintillation crystals shown in exploded view, according to one embodiment.

FIGS. 20A-20D illustrate spectrally selective crosstalk rejection layers applied to an SiPM flexi-circuit prior to folding against faces of a scintillation crystal, according to one embodiment.

FIGS. 21A-D illustrates spectrally selective crosstalk rejection layers applied to faces of a scintillation crystal prior to applying a SiPM flexi-circuit, according to one embodiment.

### DETAILED DESCRIPTION

A scintillator is a material that emits photons (e.g., in the visible spectral range) in response to stimulation by high-energy radiation. High energy radiation can refer to electromagnetic radiation having energy higher than that of ultraviolet radiation, including, but not limited to, X radiation (i.e., X-ray radiation), alpha (α) particles, gamma (γ) radiation, and beta (β) radiation. In some embodiments, the scintillator is used as a component of a radiation detector, and the light emitted by the scintillator is detected by a photodetector that is optically coupled to the scintillator.

Scintillator materials, such as scintillation crystals, can be components of radiation detectors in various apparatuses, including counters, medical gamma-ray detectors (such as those used in computer tomography (CT) or Positron Emission Tomography (PET)), security gamma-ray detectors, non-destructive testing radiography gamma-ray detectors, and industrial gamma-ray detectors used in the petroleum industry. Several metrics can be used to characterize the performance and suitability of a scintillator for various applications. Examples of relevant metrics include energy resolution, time response, light yield, stopping power, position resolution, and self-absorption. The terms scintillator and scintillation crystal are used interchangeably throughout this disclosure. The terms scintillator and scintillation crystal may be used to refer to a single crystal, a single block of other scintillator material, or combinations of multiple crystals and materials, as understood by those of skill in the art to form a scintillator or scintillation crystal. Moreover, the terms “scintillator” and “scintillation crystal” are used to broadly encompass inorganic or organic materials in single-crystal, polycrystalline ceramic, glass, plastic/polymeric, liquid, fiber, or nanocomposite form suitable to convert ionizing radiation to optical photons.

Radiation detection is utilized in various fields, including, without limitation, in medical imaging, nuclear physics, and environmental monitoring. In various embodiments, detectors are used to measure various characteristics of incident radiation particles, such as gamma rays. Examples of measured characteristics of incident radiation include the energy, incidence position, and time of incidence. One component of many radiation detectors is the scintillation crystal (e.g., a scintillator), which produces light (e.g., scintillation photons) when it interacts with the radiation (e.g., a gamma photon). The scintillation photons are detected by one or more photodetectors, such as silicon photomultipliers (SiPMs), which convert the scintillation light into an electric signal for electronic analog and/or digital processing.

The energy resolution of a scintillator detector is a measure of a scintillator detector's ability to resolve closely spaced energy levels of incoming radiation. The energy resolution can be expressed as the ratio of the full width at half maximum (FWHM) of the energy peak to its central energy (E). The energy resolution can be expressed as a percentage (e.g., FWHM/E×100%). High energy resolution enables the accurate identification and discrimination of different types of radiation. Precise energy measurements are particularly useful in applications such as PET, where energy discrimination can enhance image contrast and clarity by excluding noise events.

PET imaging relies on the detection of coincident gamma photons produced by the annihilation of positrons emitted from a radiotracer. The time response of a scintillator detector relates to its ability to precisely determine the arrival time of these photons at the detector array, which directly impacts the spatial resolution and quality of the reconstructed image.

The luminescence behavior of a scintillator is inherently time-dependent, encompassing the processes of excitation and subsequent deexcitation. Following the absorption of ionizing radiation, a finite period is required to populate the luminescent states, after which deexcitation occurs, leading to the emission of photons. This dynamic scintillation response can be modeled using a multiexponential function, which describes both the rise and fall of luminescence intensity over time. The rise and fall of luminescence intensity affect the time response characteristics of the scintillator.

The light yield of a scintillator is a metric that quantifies the number of photons emitted per unit of energy deposited by the ionizing radiation. The light yield may be measured in photons per mega-electronvolt (ph/MeV). A higher light yield implies more efficient conversion of radiation energy into visible light, which enhances the detected signal strength and improves the signal-to-noise ratio (SNR). Higher SNR is useful for achieving high-quality imaging and precise radiation quantification.

The stopping power of a scintillator material characterizes its ability to convert incoming radiation into luminescent light and/or attenuate or absorb the radiation. The stopping power is a function of the density, atomic number, and composition of the scintillator material. The selection of different scintillator materials can influence the efficiency of the scintillator in detecting different types of radiation. Scintillators with high stopping power can be used in compact scintillator designs and for applications targeting efficient detection of high-energy particles or photons.

The self-absorption of a scintillator refers to an internal phenomenon in which the light emitted by the scintillator upon excitation by ionizing radiation is partially absorbed by the material itself. This internal absorption process reduces the amount of light that escapes the scintillator for detection. Self-absorption can adversely affect the efficiency and performance of scintillator detectors. Self-absorption is primarily influenced by the overlap between the emission spectrum of the scintillator and its absorption spectrum. A substantial overlap leads to higher self-absorption, while a well-separated emission and absorption spectrum (e.g., characterized by a large Stokes shift) can minimize the self-absorption.

As used herein, the “position” of the “energy centroid” refers to the location of the intensity-weighted center of the detected scintillation energy distribution for a single interaction event. The position of the energy centroid may be based on the intensity-weighted center of the detected scintillation energy based on detection by one or more readout planes (e.g., SiPMs on one or more sides of a scintillation crystal) and/or mapped into the scintillation crystal volume by calibration. The centroid position represents an average position where the gamma-ray interacted with the scintillator and released scintillation photons. For example, a processing circuit may be used to determine the centroid position P_{c}=(x_{c}, y_{c}, z_{c}) using a center of photoluminescence method that is adapted from or analogous to a center of gravity (CoG) method, a generalization of Anger logic, which uses the energy (Ek) deposited on a photodetector k and the center position of the photodetector Pₖ=(xₖ, yₖ, zₖ).

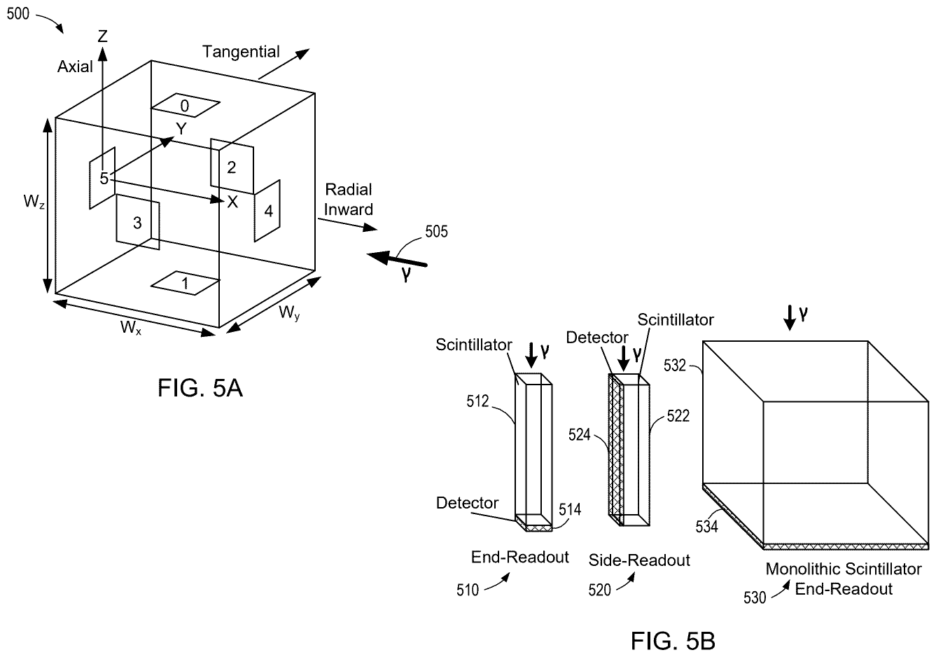

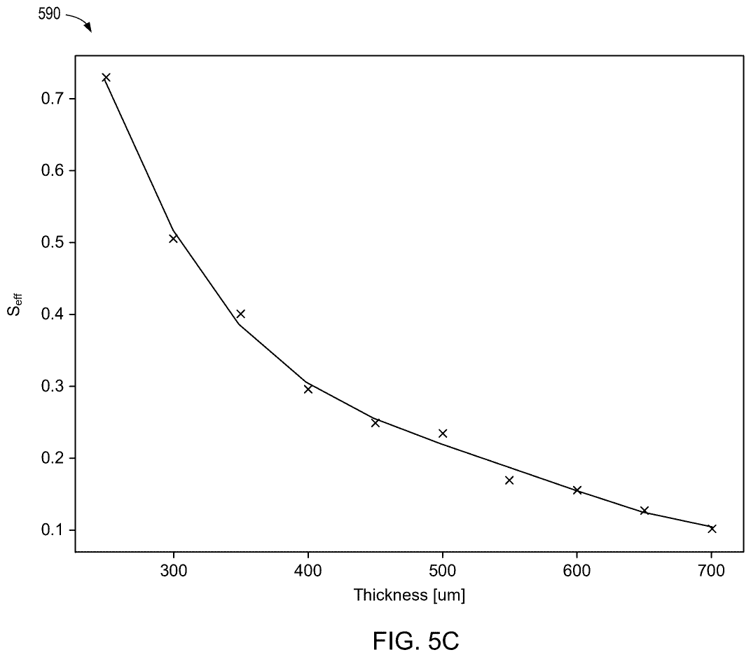

For photodetectors on the XY plane (e.g., faces 0 and 1, as defined in connection with FIG. 5A), this can be expressed as:

$${x}_{c} = \frac{{\sum }_{k}^{K} {E}_{k} {x}_{k}}{{\sum }_{k}^{K} {E}_{k}} and {y}_{c} = \frac{{\sum }_{k}^{K} {E}_{k} {y}_{k}}{{\sum }_{k}^{K} {E}_{k}} .$$

For photodetectors on the XZ plane (e.g., faces 2 and 3, as defined in connection with FIG. 5A), this can be expressed as:

$${x}_{c} = \frac{{\sum }_{k}^{K} {E}_{k} {x}_{k}}{{\sum }_{k}^{K} {E}_{k}} and {z}_{c} = \frac{{\sum }_{k}^{K} {E}_{k} {z}_{k}}{{\sum }_{k}^{K} {E}_{k}} .$$

For photodetectors on the YZ plane (e.g., faces 0 and 5, as defined in connection with FIG. 5A), this can be expressed as:

$${y}_{c} = \frac{{\sum }_{k}^{K} {E}_{k} {y}_{k}}{{\sum }_{k}^{K} {E}_{k}} and {z}_{c} = \frac{{\sum }_{k}^{K} {E}_{k} {z}_{k}}{{\sum }_{k}^{K} {E}_{k}} .$$

As used herein, a position-sensitive (or position-predicting) detector is one that, for each detected interaction, produces an event record that includes a measurement of the interaction location within the scintillator volume. The measurement of the interaction location within the scintillator volume can be expressed in one, two, or three dimensions. The scintillator detector includes a scintillation crystal that is optically coupled to one or more photosensor arrays (e.g., SiPM arrays) on one or more faces of the scintillation crystal.

The scintillator detector may further include or otherwise be associated with or connected to various processors and electronics configured to acquire per-channel waveforms or charges within a temporal gate, apply calibrations (e.g., gain or photodetection efficiency (PDE)), and/or compute a position estimate from the resulting light-spread detected at the detector array(s) on the one or more faces of the scintillation crystal. For example, the circuit components (e.g., processors, modules, discrete components, etc.) may operate to estimate the position based on (i) a centroid (center of gravity (COG) or Anger) computation with calibration weights, (ii) a maximum-likelihood or least-squares fit to a pre-measured light-spread model, (iii) a learned model (e.g., neural network or gradient-boosted regressor) trained on flood-field data to predict position from per-pixel features, or (iv) a hybrid estimator that fuses centroid, timing, and energy.

In some embodiments, a calibration may be performed to map sensor-plane features to crystal coordinates. The calibration may include gain and/or PDE equalization, spatial linearization (e.g., lookup tables), and/or bias correction using known patterns or collimated beams. The position error is the difference between the true energy centroid location and the estimated interaction location. The position resolution is the standard deviation of the unbiased position error along each axis. An equivalent figure of merit for position resolution is the full-width half-maximum (FWHM) of the position error distribution (point spread function) that is measured across the detector block.

Many examples of the presently described systems and methods are described in the context of SiPMs. However, it is appreciated that the various embodiments, solutions, examples, and architectures described herein can be used in conjunction with other types and configurations of photodetectors and photodetector arrays. For example, while many existing photodetector technologies are silicon-based and use SiPM architectures, other semiconductor materials may also be utilized. For example, a different semiconductor material may be used to form an array of avalanche photodetector architecture. As used herein, the term SiPM encompasses both silicon-based avalanche photodetector architectures and alternative architectures and/or material selections used to form functionally equivalent or functionally similar avalanche photodetector devices. As such, the presently described systems and methods may be used in conjunction with any photodetector or photodetector array technology that emits crosstalk photons at a wavelength different from the scintillation emission of the selected scintillator material.

The current technology for radiation detection often involves the use of a scintillator in optical contact with a SiPM. The scintillator produces light when it interacts with the radiation, and this light is then detected by the SiPM. Recent advancements in technology have enabled the operation of SiPMs at higher overvoltages. Higher bias voltage (e.g., a bias voltage ranging from 1-60 volts) or overvoltage (ranging from 0-36 volts) can improve the coincidence time resolution and energy resolution. However, operation at higher bias voltages also increases the dark current and the dark count rate, which is undesirable as it leads to false triggers in traditional designs. Dark counts refer to false signals in the absence of a true signal. These counts can, for example, occur due to thermally generated charge carriers, which trigger an avalanche process, and the resulting pulse mimics true photon detection signals. In addition, operating at higher bias voltages can lead to the generation of photons, often with infrared (IR) or near-infrared (NIR) wavelengths within the SiPM itself, which are referred to as crosstalk photons.

The crosstalk photons may (i) remain within the SiPM and retrigger neighboring microcells (internal optical crosstalk) or (ii) escape the crosstalk-generating SiPM, propagate through the optical stack or scintillator, and be detected by the same or a different SiPM (external crosstalk photons). Internal optical crosstalk can be substantially reduced by deep trench isolation surrounding microcells, including metal-filled or metal-lined trenches, absorptive or dielectric trench structures, or combinations thereof. Internal optical crosstalk mitigation techniques operate by optically decoupling adjacent cells within the same SiPM.

The presently described systems and methods for external crosstalk mitigation are complementary to these types of internal crosstalk mitigations. Even with internal crosstalk mitigations, such as trenching to suppress light exchange between neighboring microcells, external crosstalk photons may still exit one SiPM and reach another instrumented face. Thus, external crosstalk photons remain a significant source of noise, especially when operating SiPMs at higher overvoltages and/or bias voltages.

As described herein, the generation of external optical crosstalk photons at higher overvoltages can lead to an increased number of dark counts. These dark counts can temporarily trigger the detector, resulting in increased dead time. Furthermore, dark counts can result in a worse energy resolution, a worse position resolution, and/or a worse timing resolution, as they are noisy events that distort the true signal. Another challenge is the need for a thin interface material between the scintillator and the SiPM, especially in cases where the scintillator has SiPMs on more than one face and/or where the scintillation crystal is a monolithic scintillation crystal.

Various systems and methods are described herein to reduce optical crosstalk photons generated by SiPMs at higher overvoltages. Optical crosstalk photons are undesired secondary photons, typically in the near-infrared range. Various systems and methods are described herein to minimize the number of dark counts generated by optical crosstalk photons. Dark counts can temporarily trigger the detector, leading to increased dead time, and can also result in degradation of energy resolution, worse position resolution, and worse timing resolution, which affects the accuracy of radiation particle measurement.

This disclosure presents various systems and methods to reduce or reject external crosstalk photons. For example, a spectrally selective crosstalk rejector (e.g., a crosstalk rejection layer or embedded material) may be positioned within an optical detection path of an SiPM. The spectrally selective crosstalk rejector is substantially transparent to the scintillation photons and thus allows the SiPM to absorb the scintillation photons generated during a scintillation event. However, the spectrally selective crosstalk rejector is configured to absorb external crosstalk photons emitted by an avalanching single-photon avalanche diode (SPAD) that is external to the first SiPM (i.e., from another SiPM of the same scintillator detector or from an SiPM of an adjacent scintillator detector in a scintillator detector module).

For example, in some embodiments, a spectrally selective crosstalk rejector may be embodied as a thin optical crosstalk rejection layer in the optical detection path of a photodetector (SiPM) to block or absorb external crosstalk photons. In various embodiments, a spectrally selective crosstalk rejection layer thin film or a spectrally selective crosstalk rejection layer thin layer may be used to couple one or more SiPMs to one or more faces of a scintillation crystal. For example, the scintillator detector may be configured in a side-readout configuration or as a monolithic scintillation crystal with SiPM arrays on multiple sides. In such configurations, there are generally more crosstalk-emitting and crosstalk-receiving SiPM elements per scintillator detector. As a result, the dark count rate due to optical crosstalk photons is significantly amplified in such configurations. In contrast, in configurations in which a single SiPM is used per scintillation crystal, only a small area of the scintillation crystal is coupled to the SiPM. Consequently, fewer crosstalk photons are emitted and received per scintillator detector.

The spectrally selective crosstalk rejector rejects (e.g., absorbs) longer-wavelength optical crosstalk photons while remaining transparent to shorter-wavelength scintillation light. Most absorbing filter materials require a thickness of approximately 1 mm or more to provide sufficient optical density for filtering. However, as detailed herein, increasing the thickness of the interface between the SiPM and the scintillation crystal is undesirable. Furthermore, filter materials that are in the form of a rigid layer positioned between the scintillation crystal and the photodetector result in air gaps. Air gaps in the optical path between the scintillation crystal and the photodetectors cause a sharp refractive index mismatch, causing Fresnel reflections and reducing the number of photons that are transmitted to the photodetector (i.e., a reduction in light extraction efficiency). To mitigate this, an optical coupling medium, a grease, epoxy, or thin film with a refractive index between that of the scintillator and the photodetector may be applied between the photodetector and the optical filter, as well as between the optical filter and the scintillation crystal, to improve light extraction efficiency. However, this adds additional thickness, which is undesirable, as detailed below.

As described in greater detail below in conjunction with FIG. 5C, the effective sensitivity gain of a scintillator detector is reduced as the thickness of the spacing between adjacent detectors increases. Thus, in configurations in which lateral side-redout is used, any additional thickness between the SiPMs and the face(s) of the scintillation crystals between adjacent scintillator detectors reduces the effective sensitivity gain. Thus, for axial or transaxial side-readout configurations, any additional thickness of the interface between SiPMs and the scintillation crystal is ideally limited to a few 10's or 100's of micrometers to avoid an unacceptable level of effective sensitivity degradation.

The systems and methods described herein provide effective crosstalk photon rejection while maintaining high transparency for the scintillation light with minimal thickness. The spectrally selective crosstalk rejection layers described herein can be seamlessly integrated into a scintillator detector, adding significantly less than 1 millimeter of additional thickness, without creating any air gaps, and without requiring additional optical coupling media.

In some embodiments, an optical coupling medium may include a spectrally selective crosstalk rejection material that rejects photons of a specific wavelength, a band of wavelengths, or a set of wavelengths (e.g., one or more continuous or discontinuous wavelength bands). In various embodiments, the rejecting material enhances performance and reduces external optical crosstalk. The spectrally selective crosstalk rejection material may be referred to herein as a rejecting material, a rejection film, or a rejection layer in various contexts. The term spectrally selective crosstalk rejector is used to generally refer to layers, films, embedded materials, coatings, and the like, in various configurations and embodiments described herein.

In some embodiments, a spectrally selective crosstalk rejection material, such as a colorant (e.g., an organic dye or pigment), is dispersed within the matrix of the optical coupling medium. The refractive index of the colorant(s) may be selected to match that of the coupling medium to minimize or eliminate scattering. The refractive index of the optical coupling medium may be selected based on the refractive indices of the SiPM and scintillation crystal to improve light extraction efficiency.

In some embodiments, the spectrally selective crosstalk rejection material is applied as a thin, conformal layer on the face of the scintillation crystal and/or on a surface of the SiPM. The spectrally selective crosstalk rejection material can be deposited using various techniques, including printing, spin coating, drip casting, dip coating, drop casting, physical vapor deposition, chemical vapor deposition, atomic layer deposition, and other suitable deposition methods. These techniques enable precise control over layer thickness and distribution. For example, ultrathin films with thicknesses less than 10 micrometers may be deposited in some embodiments (e.g., 1-10 μm).

In some embodiments, the spectrally selective crosstalk rejection layer may be formed using a photoresist-based lithographic process. For example, a photosensitive polymer may be spin-coated over a target surface (e.g., SiPM passivation/window or a polished scintillator face), soft-baked (e.g., 90-120° C.), exposed through a mask, and developed to define absorber regions. The patterned resist can (i) be directly converted into an absorbing film by dye infusion, or (ii) act as a lift-off mask for depositing an absorptive layer (e.g., a dye-loaded or pigment-loaded polymer, a dielectric stack, or a nanoparticle absorber). For example, a spectrally selective crosstalk rejector may be deposited as a nanoparticle-dispersed layer.

In some embodiments, the rejecting material is integrated into the reflective material that is in contact with the scintillator on one or more surfaces not coupled to an SiPM. The reflective material may be, for example, Teflon, an enhanced specular reflector tape, or other thin reflective materials. The reflective material may be a dielectric reflector, wherein crosstalk photons are selectively transmitted to leave the detector, e.g., into a black absorbing layer. Examples of reflective materials include, but are not limited to, enhanced specular reflector (ESR) film, polytetrafluoroethylene (PTFE), and metal-coated reflectors.

In some embodiments, the optical absorption material comprises a dichroic dielectric filter configured to selectively transmit scintillation photons while reflecting or rejecting optical crosstalk photons. A dichroic dielectric filter may, for example, include multiple thin-film layers with alternating refractive indices, creating wavelength-selective interference effects that allow desired wavelengths to pass while blocking unwanted wavelengths. The dichroic filter may be applied to at least one face of the scintillator that opposes the SiPM to enhance optical filtration efficiency. In configurations where multiple SiPMs are placed on non-coplanar faces of the scintillator, an angled dichroic dielectric filter may be used to prevent optical crosstalk between these detectors. By tilting or patterning the filter layers at a non-normal incidence angle relative to the scintillator faces, the system can minimize undesired photon reflections.

In some embodiments, the rejecting material is directly incorporated into (or replaces) the cover glass or protective coating of the SiPM. This approach eliminates the need for additional interface layers and seamlessly integrates filtering capabilities into the SiPM structure. It also enables the mitigation of crosstalk photons that are emitted within the SiPM and back reflected on the cover glass or protective coating that rejection layers external to the SiPM would not be able to address. In some embodiments, the rejecting layer is coated onto the SiPM chip before a cover layer or glass is applied. In some embodiments, the rejection layer is coated onto the SiPM. In some embodiments, the rejecting material is coated on the backside of the SiPM chip. In some embodiments, the rejecting material is embedded within the scintillator itself, allowing the scintillator to perform dual functions of light emission and selective crosstalk absorption. The specific examples provided herein are provided to illustrate the functionality and ability of rejection materials to suppress external crosstalk. It is appreciated that various embodiments may include alternative configurations in which a rejection material is positioned in alternate locations in the optical path of the crosstalk photons.

According to various embodiments, the systems and methods described herein utilize a rejection layer with negligible absorption at wavelengths corresponding to the scintillator's emission, while exhibiting high optical density in the spectrum of optical crosstalk photons. The material may also be compatible with the optical coupling medium, the scintillator, and the SiPM.

The rejecting material can take various forms, including, without limitation, dyes, pigments (e.g., inorganic pigments), bulk materials, dielectrics, and plasmonic/nanocomposite absorbers. For example, dyes may be utilized with molecules specifically designed to reject photons within a targeted wavelength range or a set of targeted wavelengths or wavelength ranges. Dyes can be, for example, dispersed uniformly within a matrix, offering precise spectral selectivity and compatibility with various host materials.

Pigments may be used that comprise finely ground particulate materials that provide robust absorption and scattering properties. Pigments can be incorporated as coatings or mixed into other media to achieve wavelength-specific rejection. Bulk Materials may be used, such as solid, homogenous materials with tailored absorption coefficients. These materials can provide effective attenuation of crosstalk photons while remaining transparent to scintillation light.

Dielectrics may be used that include various materials with specialized refractive and absorptive properties, capable of selectively rejecting photons through interference effects or intrinsic absorption characteristics. Selective filtering can involve either selective transmission or selective reflection. Dielectrics can be integrated into coatings or layered structures for additional functionality.

In various embodiments, combinations of colorants (e.g., dyes, pigments, or the like), each of which may have a relatively narrow absorption profile, may be used together to attain a target absorption profile. A combination of dyes, each with a specific and narrow absorption range, may be used to create a composite material with a customized absorption profile. The resulting material is highly transparent at the wavelengths of the scintillation light, ensuring minimal attenuation of the signal, while exhibiting a sharp transition to strong absorption at the wavelengths associated with optical crosstalk photons. By leveraging the complementary properties of several dyes, a material can be formed with precise control over the absorption spectrum, effectively suppressing optical crosstalk without compromising the transmission of scintillation photons. For example, in some embodiments, a polymer matrix includes two or more dyes with different spectral absorption ranges, such that a cumulative spectral absorption range of the polymer matrix exceeds that of any one dye by itself.

Various embodiments of the systems and methods described herein utilize a monolithic scintillator and detector configurations with multiple SiPMs. A monolithic scintillator or monolithic scintillation crystal may include a block or blocks of scintillating material with an unsegmented optical clarity larger than a spatial detection pitch. For example, a monolithic scintillator may include a single block of material that allows photon interactions to occur uniformly throughout its volume. The monolithic scintillator may be a continuous or functionally continuous block of scintillating material. The monolithic scintillator may be fabricated from a single piece of scintillating material. In other embodiments, a monolithic scintillator may be fabricated from multiple pieces of scintillating material that are substantially identical and bonded together to preserve the optical and physical properties, thereby behaving as a single, continuous scintillator.

In other embodiments, a monolithic scintillator or monolithic scintillation crystal may include a block of scintillating material in which photon interactions occur nonuniformly throughout the volume. The approaches, calculations, and estimates described herein can be adjusted based on a known, non-uniform photon interaction response of the volume. In some embodiments, a monolithic block may comprise two different scintillator materials, different dopants within different regions of the block, quantum dots, impurities, or other non-uniform characteristics or materials. In such embodiments, the monolithic scintillator may still exhibit an unsegmented optical clarity that exceeds the spatial detection pitch.

The terms “monolithic scintillation crystal” and “monolithic scintillator” are used interchangeably herein to refer to a functionally continuous volume of scintillating material, regardless of whether it is a single piece of material or multiple pieces of material joined or bonded together. In some instances, a “monolithic scintillator” is referred to as a “scintillator” for simplicity, as can be contextually understood. The precise localization of photon interaction points within a monolithic scintillator can be performed by analyzing the distribution of scintillation light over a photodetector array.

The presently described systems and methods are particularly useful for monolithic scintillator configurations with SiPMs on multiple faces. Due to the large SiPM detector area in monolithic detectors, more optical crosstalk photons are emitted, which degrades the operation of monolithic scintillators. In configurations with a monolithic scintillator and SiPM arrays on multiple sides, there is both a larger number of SiPMs per scintillation volume that can emit external crosstalk photons and more SiPMs to receive the optical crosstalk photons. Accordingly, the light extraction efficiency is higher, which results in an overall amplification of the dark count rate due to external crosstalk photons.

As previously described, reflective materials may be applied to the sides of the scintillator that are not in optical contact with the SiPMs. The reflective surfaces enhance light collection efficiency, improving spatial resolution, energy resolution, and coincidence timing resolution (CTR). However, the increased light reflection also increases optical crosstalk by redirecting photons toward the SiPMs, potentially generating false signals. The amount of optical crosstalk can be reduced as described herein, thereby enabling the operation of monolithic radiation detectors at lower dark count rates.

The external crosstalk detected by the SiPMs of a scintillator detector scales with the total avalanche activity, including from both true scintillation events and dark counts, multiplied by the light-extraction/capture efficiency (LEE) of the SiPM (or other photodetector device). Multi-sided readout differs fundamentally from single-sided designs for various reasons, including that (i) there are many more SPADs emitting crosstalk photons and dark-trigger avalanches, and (ii) cross-face capture greatly increases LEE. The cross-face capture greatly increases the LEE (as compared to a single-sided configuration) because photons emitted by an SiPM on one face can be collected by a SiPM on another face of the same scintillator detector. Thus, the potential for external crosstalk is higher in multi-sided detection configurations due to both emission conditions (more SiPMs and SPADs) and capture conditions (higher LEE via cross-face paths).

In some embodiments, a multi-sided scintillator detector with SiPM arrays on more than one face employs an ultrathin, absorptive, conformal spectrally selective crosstalk rejection layer on each face of the scintillation crystal that has SiPMs thereon (i.e., each SiPM-instrumented face). In such embodiments, external crosstalk photons that might be detected by one of the SiPMs (i) first, exit the emitting face and (ii) second, enter a detecting face. By coating each SiPM-instrumented face, each external crosstalk photon encounters two rejection layer passes before possible detection.

The selective crosstalk rejection layer can be described as exhibiting an optical density OD_XT in the crosstalk band. The single-pass crosstalk transmittance of the selective crosstalk rejection layer can be expressed as T=10^{-OD_XT}, and the two-pass cross-face transmittance can be expressed as T=10^{-2*OD_XT}. By contrast, scintillation photons originating within the crystal (i.e., resulting from a scintillation event of received gamma radiation) and traveling to the nearest readout face only pass through the selective crosstalk rejection layer once before being detected by an SiPM. The selective crosstalk rejection layer has negligible or relatively small attenuation at the wavelengths corresponding to the scintillation photons. Accordingly, the single-pass transmission remains high and the added haze is minimal. In such configurations, the systems and methods described herein incorporate a selective crosstalk rejection layer with an asymmetric pass count, where there are two passes for external crosstalk photons but only one pass for scintillation photons. The asymmetric pass count increases selectivity, such that cross-face crosstalk is strongly attenuated by two passes through the selective crosstalk rejection layer; yet, the information-bearing scintillation photons are preserved and only pass through the selective crosstalk rejection layer once.

In various embodiments, the SiPM(s) of a detector may be operated at high overvoltages. Operating the SiPMs at high overvoltages can provide higher photodetection efficiency and better energy resolution. Operating at higher overvoltages would theoretically improve coincidence time resolution; however, at higher overvoltages, the optical crosstalk caused by the increased dark count rate results in a deterioration of the coincidence time resolution. The presently described systems, methods, and architectures are proposed to enable improved coincidence time resolutions at higher overvoltages by suppressing external crosstalk.

In many traditional approaches, a maximum operational voltage or even a maximum operational overvoltage is limited by the degradation caused by the increased dark count rate. However, in the presently described systems and methods, the optical crosstalk is reduced via the crosstalk rejection (e.g., rejection materials, such as scattering materials, absorbing materials, filtering materials, and the like), which allows for the operation of SiPMs at higher overvoltages than normally considered acceptable, without the deterioration in performance and/or with increased performance. Various approaches for incorporating a filter and/or reject material are contemplated herein, including embodiments in which the selective crosstalk rejector is part of, on, or in the SiPM, applied as an optical coupling medium, added as an interstitial rejection layer between the SiPM and the scintillator, and/or applied to one or surfaces or faces of the scintillator.

For instance, as described herein, a thin film or ultrathin film rejection layer may be applied directly to the silicon of the SiPM, integrated as a layer or particles (e.g., a dye) within a coupling layer between the SiPM and a cover layer, integrated within the cover (e.g., within a protective cover glass or polymer), and/or as a layer on top of the cover. In various embodiments, the rejection layer may be formed as a layer or particles within a layer used to form a coupling medium or interstitial rejection layer that can be applied or otherwise positioned between the SiPM and the face of the scintillator. In some embodiments, the rejection layer may be formed as a layer on the photodetector surface, as a distribution of particles (e.g., dye or pigments) within a region of the bulk crystal of the scintillator itself, as a layer within the scintillation crystal, and/or integrated within or as a layer on the reflective materials applied to the surfaces of the crystal that lack an SiPM interface.

The embodiments of the systems and methods provided within this disclosure are not intended to limit the scope of the disclosure but are merely representative of possible embodiments. Additionally, the steps of a method do not necessarily need to be executed in a specific order, or even sequentially, and they do not need to be executed only once. Numerous specific details are provided in the following description of the figures to offer a thorough understanding of the possible variations of the systems and methods. However, the disclosed concepts, systems, methods, apparatuses, etc., may be practiced without some or all the specific details provided and illustrated in the figures. For clarity, technical material that is known in the technical fields related to this disclosure is not described in detail.

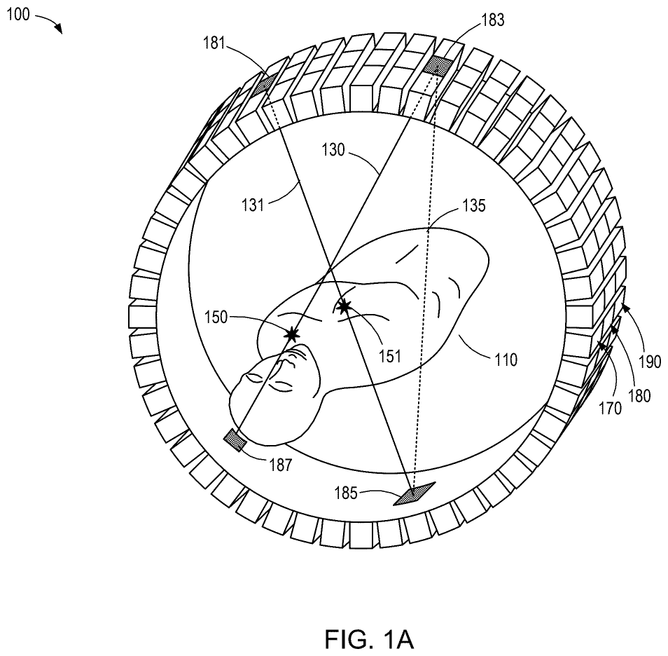

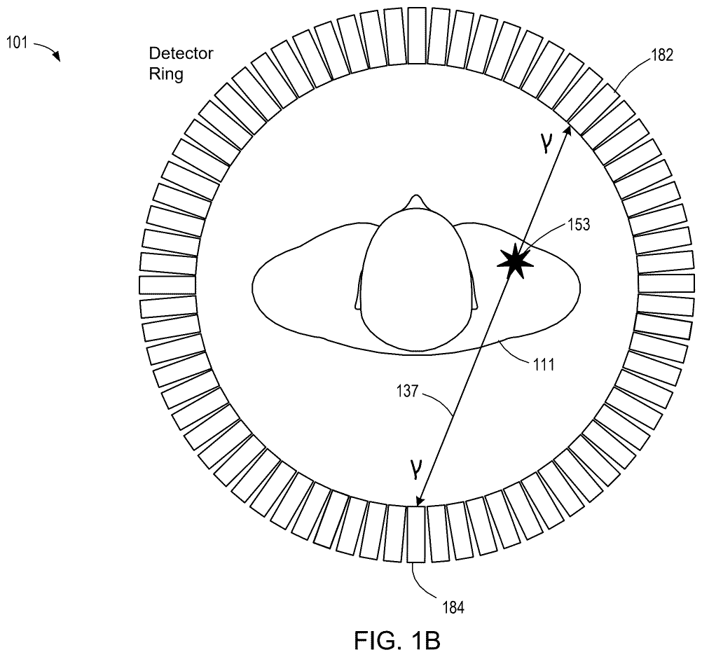

FIG. 1A illustrates a perspective view of a PET detector system 100 and positron sources 150 and 151 within a patient 110 (illustrated as a human torso), according to one embodiment. In the illustrated embodiment, the PET detector system 100 includes three rings of gamma radiation detector modules. The first ring 170 of gamma radiation detector modules is closest to the viewer, the second ring 180 of gamma radiation detector modules is in the middle, and the third ring 190 of gamma radiation detector modules is farthest from the viewer. In the illustrated example, each ring 170, 180, and 190 includes forty-eight gamma radiation detector modules. The number of detector modules and the number of rings of detector modules may vary based on a particular application, cost considerations, target size, target resolution, etc.

The positron sources 150 and 151 represent boluses of positron activity that result in annihilation radiation in which two opposing gamma photons are emitted in opposite directions. The opposing gamma photons emitted by the positron source 150 are detected by detector modules 183 and 187. A processor or other circuitry can use the known detector location and measured arrival times of the gamma photons detected by detector modules 183 and 187 to compute the line-of-response (LoR) 130 and determine the location of the positron source 150. The processor or other circuitry may identify a detected gamma photon as being a salient annihilation event based on the total detected energy being equal to or within a threshold range of the expected 511 keV.

The opposing gamma photons emitted by the positron source 151 are detected by detector modules 181 and 185. The processor or other circuitry can use the known detector location and measured arrival times of the gamma photons detected by detector modules 181 and 185 to compute the line-of-response 131 and determine the location of the positron source 151. As discussed in greater detail below, if the positron sources 150 and 151 emit their respective pairs of opposing gamma photons at the same time (or nearly the same time), there is a risk that the processor or other circuitry may erroneously compute a line-of-response 135.

Throughout this disclosure, the detector modules are described and illustrated as being used in a detector ring (e.g., a ring of detector modules). The scintillation crystals in each detector module have a distal end-face that faces into the detector (e.g., into the ring, wherein the distal end faces are located on the inner diameter of the ring) to receive a gamma photon. However, it is appreciated that a detector ring may include detector modules in the shape of a ring without forming a complete or full ring (e.g., with only the top and bottom detector modules in place). Moreover, a detector may be embodied as two (or more) opposing panels (e.g., flat, convex, or concave panels) of detector modules that act in concert to detect annihilation events. For example, a detector may include a first planar panel of detector modules and a second, opposing planar panel of detector modules. A subject may be positioned within the detector by being placed between the two opposing planar panels. As such, a detector module may include a plurality of scintillation crystals (e.g., in a one-dimensional or two-dimensional array) that have distal ends oriented into the detector (e.g., into the cavity or space between the opposing panels, such that the distal end faces are located on the inner diameter of the ring).

FIG. 1B illustrates a side view of another example of a PET detector system 101 and a positron source 153 within a human 111, according to one embodiment. In the illustrated embodiment, the PET detector system 101 includes a ring with seventy-two gamma radiation detector modules. The opposing gamma photons emitted by the positron source 153 are detected by detector modules 182 and 184. A processor or other circuitry can use the detector module locations and arrival times of the gamma photons detected by detector modules 182 and 184 to compute the line-of-response 137 and determine the location of the positron source 153. Each of the detector modules may include a scintillation crystal.

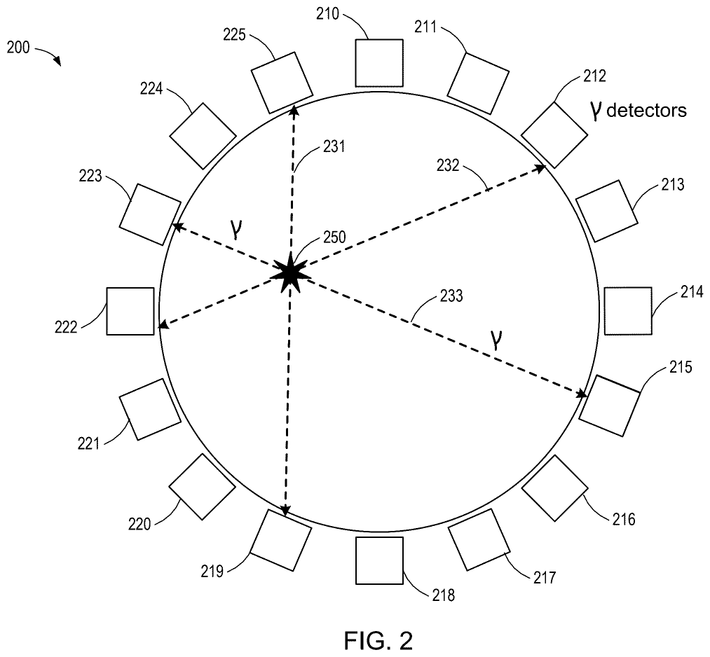

FIG. 2 illustrates a block diagram of a PET detector system 200, according to one embodiment. The simplified block diagram includes a single ring of sixteen gamma radiation detector modules 210-225. Multiple lines-of-response 231, 232, and 233 are shown that intersect a bolus 250 of positron activity, according to one embodiment. Annihilation radiation from the bolus 250 of positron activity generates numerous simultaneous emissions of equal and opposite gamma photons. The system 200 can determine a range for a positron's point of origin along the line-of-response 233 extending between its two detection points (e.g., within detector modules 223 and 215).

Additional lines-of-response 231 and 232 are computed during a measurement period in which subsequent annihilation events occur within the bolus 250. A period of time during which annihilation events are detected from the bolus 250 is referred to as a scan. The system 200 is used to capture a scan that provides multiple lines-of-response for localizing a positron matter's point or region of origin. A PET scanner (e.g., including or embodied as a PET detector system as described herein) may use the ring of detector modules 210-225 to develop images of varying concentrations and locations of positron material without the use of shielding collimators, such as those employed in single photon emission computed tomography (SPECT), which reject oblique angles of origin. In other embodiments, a PET scanner may utilize axial collimators, referred to as septa, between multiple detector rings to reduce scatter radiation originating from the field of view (e.g., within a human body). However, other PET scanners operate in “3D” mode, without septa, given faster scintillators and advancements in electronic processing, which can handle larger event rates, leading to enhanced system sensitivity.

Annihilation radiation's dual gamma events are discriminated against (e.g., identified relative to) a multitude of other unrelated gamma events (generally referred to as “singles events”) through the use of a time-based coincidence window. The coincidence window, which may be, for example, less than five nanoseconds (e.g., two nanoseconds), is used to reject singles events that could not have occurred from a corresponding single annihilation within the field of view, given the scanner's bore diameter (corresponding to the diameter of the ring of detector modules 210-225) and the propagation speed of gamma radiation. For example, for a PET scanner with detector modules in a ring with a diameter of 70 centimeters (e.g., a bore diameter of 70 centimeters), a positron annihilation in the center of the bore would necessitate approximately 1.167 nanoseconds for each gamma photon, traveling at the speed of light (29.98 centimeters per nanosecond), to arrive within a scintillator of one of the detector modules in the ring. Correspondingly, a positron annihilation located along the circumference of the bore would necessitate twice this amount of time for one of the gamma photons to scintillate into a detector module on the opposite side of the ring. As such, a coincidence window, along with select pairings of detector modules comprising only reasonable geometric lines-of-response, reduces uncorrelated gamma events from being otherwise recorded as coincident.

When two gamma events meet the criteria for being recorded as a singular coincident event (e.g., within the time window and detected by appropriate detector modules), such an event is referred to as a “prompt” event. Prompt events may still yet be comprised of two uncorrelated and independent gamma events originating from different annihilation events or other radiation sources, which are referred to as “random” coincident events. However, when two gamma detections authentically originate from a single positron annihilation, then such an event is referred to as a “true” coincident event. Hence, “true” coincident events occurring from bona fide positron annihilations are equated to the number of “prompt” coincident events (e.g., coincident events from an unknown source) less the number of “random” coincident events (coincidence events known or statistically determined to be random singles measured within the coincidence window).

In various embodiments, a PET scanner may further filter “scatter” events, which result when a gamma photon partially deposits its energy into a nearby material and then propagates its remaining energy into a detector module. Such scatter events are typically filtered since their point of origin is uncertain, given their possible deflection. Scatter events usually terminate in a detector with substantially less energy than their original 511 keV. Therefore, filtering may involve measuring the amount of energy deposited by a gamma event and rejecting such events that fall below a lower-level-discrimination (LLD) threshold. Additionally, upper-level discrimination (ULD) may also be utilized to reject any near-simultaneous accumulation of multiple events into a detector or the possibility of a single higher-energy nuclear particle deposition. According to various embodiments, a PET scanner may utilize an LLD threshold value between 325 keV and 511 keV and a ULD threshold value between 511 keV and 675 keV.

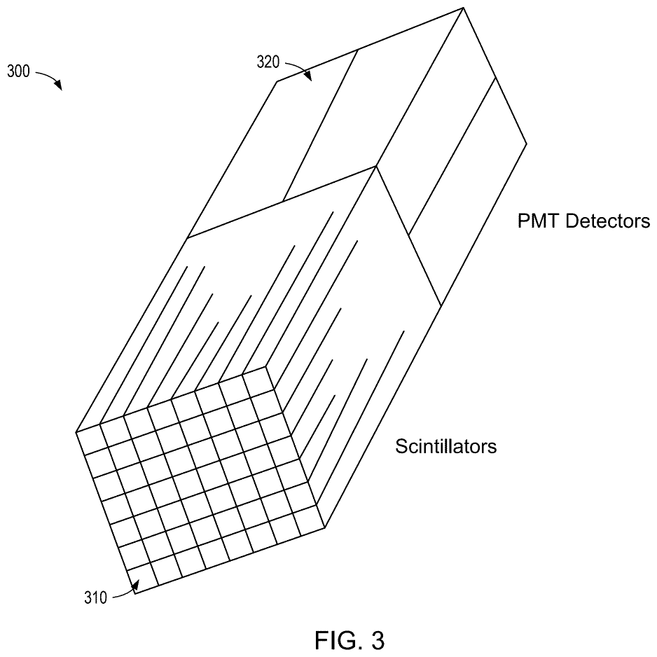

FIG. 3 shows a tube-based detector module 300 with four photomultiplier tubes 320 (PMTs) in a quadrant-based detection subsystem, according to one embodiment. The four photomultiplier tubes 320 are connected to a two-dimensional array of elongated scintillation crystals 310. The example two-dimensional array of elongated scintillation crystals 310 includes 56 elongated scintillation crystals in a 7×8 array. Some embodiments of gamma detector modules for PET scanners include inorganic scintillators (e.g., Bismuth Germanate, Bi₄Ge₃O₁₂, referred to as BGO, or Lutetium Oxyorthosilicate, Lu₂(SiO₄)O, referred to as LSO, Lu_{2(1-x-y)}Y₂ₓSiO₅, referred to as LYSO, etc.) coupled to a light guide and connected to a quadrant of photomultiplier tubes. Examples of scintillation crystals include, but are not limited to, lutetium oxyorthosilicate (LSO), lutetium-yttrium oxyorthosilicate (LYSO), lutetium-gadolinium oxyorthosilicate (LGSO), bismuth germanate (BGO), and cesium iodide (CsI).

The illustrated configuration may be referred to as a block detector module. A detector module may comprise one or more block detectors. The block detector, in this case, shares a channel of electronics for processing nuclear gamma singles events from multiple elongated scintillation crystals 310. The elongated scintillation crystals 310 may be pixelated, as illustrated, into a plurality of elongated scintillation crystals 310, which may be etched, polished, and/or wrapped in a reflective material. Pixelation of the elongated scintillation crystals 310 into an array of elongated rectangular prisms facilitates determining the position of an impinging gamma photon within the rectangular area of an end-face of each elongated scintillation crystal 310.

In various embodiments, select areas or faces of each elongated scintillation crystal 310 may be wrapped or coated with a reflective material. The utilization of four photomultiplier tubes 320 allows for the collection of light output from any given crystal to be ratiometrically measured to determine which crystal underwent gamma interaction, thereby locating such an event on an X-Y plane for the detector face.

The sharing of processing electronics for singles events on a block basis is motivated by cost limitations, reduced power consumption and dissipation, and small packaging volume. In the block processing configuration, the detector module 300 exclusively processes a singles event via the block's processing electronics, while any other incident radiation within the same block's processing time is ordinarily rejected. Such processing time is referred to as detector dead-time. The detection area of a defined block is of consideration for PET since sharing processing electronics for too large a block may curtail count-rate ability and sensitivity, while too small a block may drive costs exceedingly high and complicate thermal considerations for practical volumes.

In various embodiments, solid-state photosensors (e.g., silicon-based photosensors) may be used instead of photomultiplier tubes. Conventional semiconductor-based PIN photodiodes and/or avalanche photodiodes (APD) produce an analog signal proportional to incident photonic flux. However, APDs result in a gain from impact ionization occurring from their high reverse bias, wherein generated electron-hole pairs further collide with nearby atoms, liberating additional charge carriers beyond the initially generated photocurrent. Although APDs typically provide much better sensitivity, they commonly possess higher noise levels than PIN photodiodes. More recent developments have resulted in the single-photon-avalanche-photodiode (SPAD), which operates at very high reverse bias, commonly referred to as Geiger-mode. SPADs are optimized for single photon detection, which triggers a self-sustaining avalanche breakdown that must be quenched during operation. As a result of this self-sustaining breakdown, SPADs do not provide information on light intensity like that of APDs or PIN photodiodes. However, SPADs may be configured in parallel with large numbers of other SPADs and referred to, on the whole, as a silicon photomultiplier (SiPM) photosensor. An SiPM results in a unique photosensor that produces a signal proportional to light intensity, resulting from the corresponding number of triggered SPAD cells. In this manner, the SiPM behaves as an analog photon counter whereby each individually triggered SPAD contributes to the total photocurrent output of the sensor and results in a much higher gain than that of a single APD or a PIN photodiode.

SiPM photosensors, which may include tens, hundreds, or even thousands of single-photon avalanche photodiodes per square millimeter, are placed onto a single silicon substrate. Silicon photomultipliers may be used where timing resolution and photonic dynamic range are both required, desired, and/or otherwise determined to be useful in a specific application or use case. Advantages of such devices over photomultiplier tubes include their reduced packaging volume, reduced bias voltage, reduced sensitivity to magnetic fields, and improved single-photon timing resolution. Disadvantages of silicon photomultiplier versus conventional photomultiplier tubes include higher dark currents, or counts, for a given temperature and area, as well as complex multi-exponential pulse shapes. Solid-state photosensors can be used in place of photomultiplier tubes on a one-to-one basis within a block or can be coupled one-to-one to each elongated scintillation crystal within a block.

As used herein, references to a “photosensor,” “photodiode,” and/or “photodetector” are not meant to be restrictive or exclusive to any particular embodiment unless explicitly stated otherwise. The term “readout,” as used herein, refers to the measurement, survey, assessment, or processing of the topic referenced. Similarly, the use of “lateral” readout, “side” readout, and “side” sensing all generally refer to the photo-sensing of a scintillator where a transducing face of the photosensor is coupled to a scintillator face parallel to a transaxial direction of the scanner's bore and, hence, not taken from either end-face parallel to the scanner bore's axis. Additionally, as used herein, “scintillator,” “crystal,” “pixel,” and “block” may each refer to the scintillator material configured in different forms.

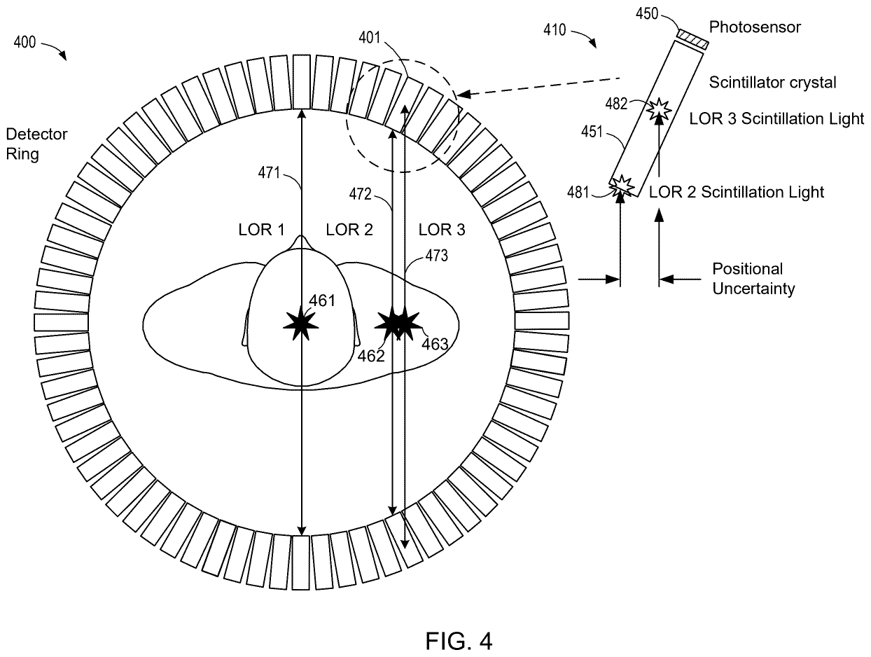

FIG. 4 is a block diagram of PET scanner 400 with a ring of detector modules 401 and multiple lines-of-response 471, 472, and 473, according to one embodiment. A positron annihilation 461 from the center of the ring results in the line-of-response 471 that orthogonally impacts the end-face of the elongated scintillation crystal of a pair of detector modules. Positron annihilations that occur off-center, such as positron annihilations 462 and 463, result in gamma photons that impinge upon scintillators at an oblique angle, resulting in a parallax effect. As shown in the exploded view of an elongated scintillation crystal 410 of a detector module 401 (in the upper right corner of the drawing), two gamma events 481 and 482 scintillate within the same elongated scintillation crystal 410 yet originate from separate locations (at positron annihilations 462 and 463, respectively) within a patient's body. An end-face photosensor 450 on the proximal end-face of the elongated scintillation crystal 410 detects the scintillation photons generated by the elongated scintillation crystal 410 in response to the received gamma photon.

The true lines-of-response 472 and 473 for the annihilation events 462 and 463 are illustrated. However, these two gamma scintillation events 481 and 482 within the same crystal 410 may be processed such that the two different positron annihilation events 462 and 463 are detected as originating from the same position, thus resulting in imaging errors. Such a parallax effect results in geometric uncertainty, including the further possibility for a random event, given that some embodiments of detector module 401 cannot discern between an event normal to the face of an elongated scintillation crystal 451 versus that arising from a severe oblique angle. The position of the end-face photosensor 450 on the proximal end-face of the elongated scintillation crystal 451 allows the end-face photosensor 450 to detect scintillation photons generated within the elongated scintillation crystal 451, but without the ability to determine a depth-of-interaction. For example, the end-face photosensor 450 does not distinguish between scintillation events 481 and 482.

FIG. 5A illustrates a scintillation crystal 500 with an overlaid coordinate system and face numbering, according to one embodiment. The scintillator dimensions are expressible as Wx×Wy×Wz, which correspond to the x, y, and z axes, respectively. Each scintillator face is labeled with a number from 0 to 5. In the illustrated example, the x-axis corresponds to a radial axis oriented into a detector ring, the y-axis corresponds to a tangential axis or (transaxial axis) tangent to the detector ring, and the z-axis corresponds to the axial axis extending along the axis of the detector ring.

The direction of the gamma-ray photon 505 is modeled in the negative x-direction relative to the numbered faces or “sides” 0-5 of the scintillation crystal 500. The inner end-face (labeled as face “4”) is radially oriented into a detector ring to receive the gamma-ray photon 505. The outer end-face (labeled as face “5”) is opposite the inner-end-face 4 and is radially oriented outward relative to the detector ring. Opposing lateral faces labeled “1” and “0” are axially oriented with respect to the detector ring, and opposing lateral faces labeled “2” and “3” are tangentially oriented with respect to the detector ring. The numeric labeling of the faces of the scintillation crystal 500 is for convenience only, and it is appreciated that other labeling or descriptive identifiers may be utilized. In the illustrated example, the monolithic scintillation crystal 500 is a cuboid, such that each face is orthogonal to other edge-connected faces along one of the axes. For example, face 5 is orthogonal to faces 0, 1, 2, and 3. In the illustrated example, the scintillation crystal 500 is a cube where the dimensions Wx=Wy=Wz.

In some instances, the configurations of scintillation crystals and photodetector arrays are named based on the faces or sides that include photodetector arrays. For example, a scintillation crystal with photodetector arrays on faces 0, 2, and 5 is referred to as a scintillator detector with a Side025 configuration, where faces or sides 1, 3, and 4 have reflective material but no photodetector arrays. As another example, a Side0125 configuration includes four photodetector arrays on each of the faces 0, 1, 2, and 5. A scintillator detector comprising a scintillation crystal with photodetector arrays on all six faces would be referred to as a Side012345 configuration.

FIG. 5B illustrates simplified block diagrams of various scintillator detector readout configurations, according to various embodiments. The end-readout configuration 510 includes a photodetector, or a photodetector array, 514 along an outer end-face of an elongated scintillation crystal 512. The side-readout configuration 520 includes a photodetector array 524 along a lateral face of an elongated scintillation crystal 522. The monolithic scintillator end-readout configuration 530 includes a photodetector array 534 along an outer end-face of a monolithic or block scintillator 532.

FIG. 5C illustrates a graph 590 of the relative effective sensitivity of lateral side readout configurations for various thicknesses of lateral detectors, according to one embodiment. The vertical axis shows the relative effective sensitivity gain, S_{eff}. As illustrated, the thickness of the spacing between neighboring scintillator pixels can negatively affect the effective sensitivity gain (S_{eff}) of the PET scanner. While graph 590 is intended to show the effective sensitivity gain as a function of spacing between scintillators in side-readout configurations, the concept is equally applicable to configurations that have spacings in the tangential or axial direction with side-readout SiPMs (e.g., configurations in which SiPMs are positioned or disposed on any of sides 0, 1, 2, 3 of FIG. 5A). In contrast, additional spacing due to the thickness of SiPMs and other materials on the radial end-faces (sides 4,5 in FIG. 5A) does not lead to a larger spacing between scintillators. For purposes of side-readout configurations, the thickness values along the horizontal axis of graph 590 include the thickness of the SiPM, any interposer or other electrical components, as well as the thickness of any optical coupling medium, filter layers, rejection layers, and/or any other material between the SiPM and the face of the scintillation crystal. As illustrated, there is a sharp drop in effective sensitivity gain, S_{eff}, with every 100 micrometers of additional thickness between neighboring side-readout detector components.

Thus, it is readily appreciated that the interface thickness between an SiPM and the face of the scintillation crystal may only be a few 10's or 100's of micrometers thick before unacceptably degrading the effective sensitivity gain. Accordingly, for detectors with SiPMs on the tangential or axial sides, spectrally selective crosstalk rejection layers added to reduce external optical crosstalk (i.e., crosstalk photons from other SiPMs, including other SiPMs in the same detector module and/or SiPMs in neighboring detection modules) should add additional thickness or spacing of much less than 1 millimeter between neighboring scintillator-detector components to maintain sufficiently high effective sensitivity gains.

The systems and methods described herein provide external crosstalk photon rejection while maintaining high transparency for the scintillation light with minimal thickness. The spectrally selective crosstalk rejection layers described herein can be seamlessly integrated into the scintillator/photodetector system without causing air gaps or requiring additional optical coupling media.

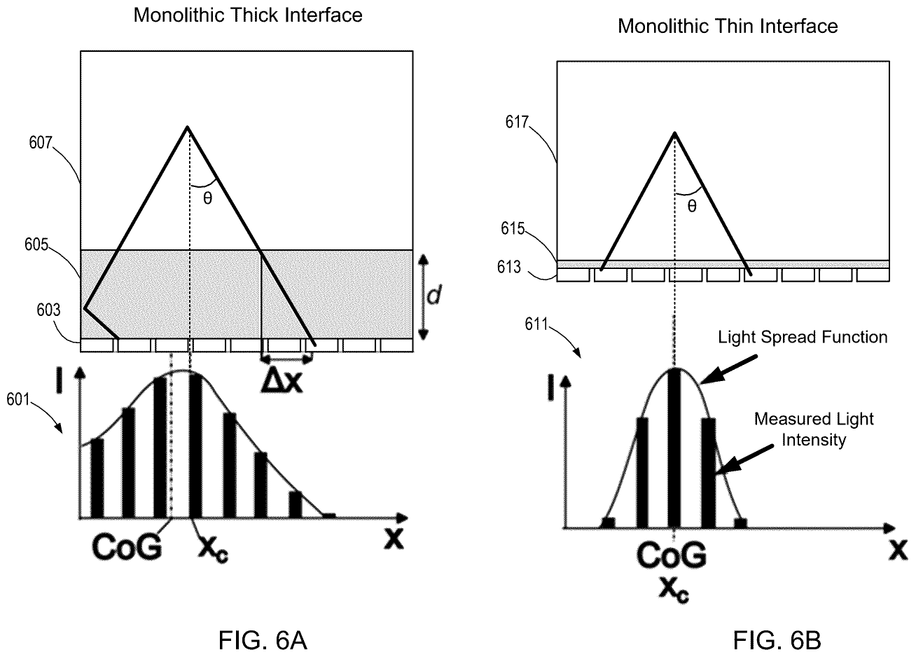

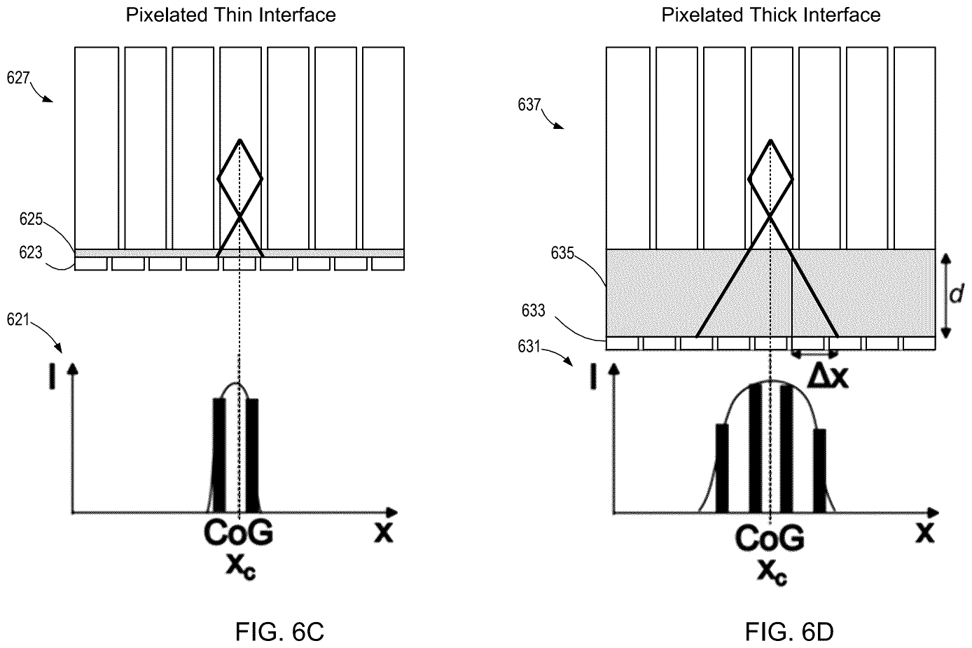

FIGS. 6A-D illustrate examples of sensor-plane light-spread for scintillator detectors with monolithic scintillation crystals and pixelated scintillation crystals, including examples with thin interfaces and thick interfaces, according to various embodiments.

FIG. 6A illustrates the sensor-plane light spread 601 with a thick interface layer 605 between the SiPMs 603 and a monolithic scintillation crystal 607, according to one embodiment. The example thick interface layer 605 may have a thickness of approximately 1 millimeter. The thick interface layer 605 acts like a waveguide, where off-normal signal rays traverse the interface layer 605 of thickness, d, at an internal angle θ and undergo a lateral position walk-off Δx=d*tan θ before reaching the SiPMs 603. The escape cone of rays that can transmit from the scintillation crystal 607 (refractive index nₛ) into the interface layer 605 (refractive index nᵢ) is bounded by the critical angle θ_{s,max}=arcsin(nᵢ/nₛ), for any transmitted rays with incidence angles θₛ≤θ_{s,max}.

Furthermore, in embodiments in which the interface layer 605 comprises multiple layers with different refractive indices, a conservative bound on the escape cone is obtained by using the smallest refractive index in the stack, since the transmission must be feasible across every interface and the lowest-index layer imposes the tightest constraint.

Because Δx scales linearly with the thickness, d, of the interface layer 605, additional thickness widens the point-spread symmetrically to the left and right by ±Δx (total added width 2Δx for a ray pair±θ). Using LYSO with a refractive index of approximately 1.82 (nₛ≈1.82) as an example, and an interface/coupling medium with a refractive index of 1.4 (nᵢ=1.40), the escape-cone limit in such a scintillator detector is approximately 50.3 degrees (θ_{s,max}≈50.3°). The resulting position walk 2Δx for an interface layer that is one millimeter thick is 2.41 mm.

The thick interface layer 605 results in a broader light spread at the sensor plane (e.g., at the SiPMs 603), reducing spatial gradients in the light spread and increasing estimator variance for center-of-gravity, Anger-logic, maximum-likelihood, learned, or other position estimators. The position estimator infers the interaction location from spatial contrasts in the detected light-spread across pixels. As the light-spread (point-spread function, PSF) becomes wider, those contrasts (i.e., the spatial gradients of expected pixel signals) become smaller, so a given amount of measurement noise produces a larger uncertainty in the inferred position and a worse position resolution. Furthermore, the thick interface layer 605 increases the likelihood of additional side-wall reflections, as shown on the left side of the thick interface layer 605. Reflections can distort the symmetrical shape of the light-spread function, causing the center of gravity (CoG) of the light-spread function to deviate from the position of the energy centroid, x_{c}. This not only increases the variance of the position prediction (i.e., resulting in a larger position resolution) but also causes the position prediction to be calculated with increased error.

For the reasons detailed above, the optical interface between the scintillation crystal 607 and the SiPMs 603 should be kept as thin as practicable to preserve the information-bearing light-spread. Because the lateral walk-off scales as Δx=d*tan θ, millimeter-scale elements, such as rigid filters, thick windows, and/or thick couplants, broaden the light spread by hundreds of micrometers to several millimeters.

FIG. 6B illustrates the sensor-plane light spread 611 with a thin interface layer 615 between the SiPMs 613 and a monolithic scintillation crystal 617, according to one embodiment. The thin interface layer 615 may be, for example, a conformal rejection layer deposited according to any of the various embodiments described herein. The interface layer 615 may have a thickness of, for example, two microns. The position walk for the 2-micron-thick interface layer 615 is only 2Δx≈0.005 mm. Thus, for the same transmitted angle within the escape cone, a millimeter thick interface layer 605 (FIG. 6A) in the sensor plane causes an additional light-spread by millimeters (comparable to a pixel pitch), whereas the disclosed few-micrometer thick conformal interface layer 615 (FIG. 6B) adds only a few micrometers in position walk which is much smaller than conventional SiPM pitches. Accordingly, the thin interface layer 615 does not negatively impact the performance of the energy-centroid position estimation.

As described herein, the interface layer 615 may consist of or comprise a thin, spectrally selective crosstalk rejection layer. The thin spectrally selective crosstalk rejection layer preserves the native, information-bearing light-spread of scintillation photons while removing the long-wavelength external crosstalk photons, yielding a narrower light spread with improved position resolution. In various embodiments, the total interfacial stack thickness, d, between the photosensitive elements (e.g., SiPMs) and the scintillator (e.g., scintillation crystal), including the sum of any window, filters, couplant layers, rejection layers, etc., is selected so that the position walk contribution from the interface stack is small compared to the blur from pixel sampling. As an example, the stack thickness, d, may be selected to satisfy 2d*tan θ_{s,max}≤αP, where

$${θ}_{s , max} = arcsin ( \frac{{η}_{i}}{{n}_{s}} ) ,$$

where ηᵢ is the smallest refractive index of a material in the interposed optical stack between the face of the scintillation crystal and the SiPM In various embodiments, α may be selected between 0 and 0.5 or between 0 and 1 to ensure that the interface-induced broadening caused by the spectrally selective crosstalk rejection layer is constrained to no more than a fraction α of the pixel pitch, P.

FIG. 6C illustrates the sensor-plane light spread 621 with a thin interface layer 625 between the SiPMs 623 and an array of pixelated scintillation crystals 627, according to one embodiment. As described above in conjunction with FIG. 6B, the thin interface layer 625 maintains positional walk to a negligible value.

FIG. 6D illustrates the sensor-plane light spread 631 with a thick interface layer 635 between the SiPMs 633 and an array of pixelated scintillation crystals 637, according to one embodiment. In multi-sided readout configurations, thin interfaces may be even more significant since any added spread and asymmetry accumulates across faces, degrading position linearity and resolution. Using LYSO as an example, assuming a θ_{s,max} of approximately 50.3°, and an example SiPM pixel pitch of P=3 mm, the maximum allowable interface thickness, dₘₐₓ, for an α=0.5 is 0.62 mm (dₘₐₓ=0.62 mm). The value of α can be selected to maintain interface-induced blur to a fraction of a pixel. Selecting a value α≈0.5 maintains the interface-induced blur to less than half of a pixel, which under quadrature addition of independent blurs raises the point spread function width, and associated position resolution, by about 22%. (e.g., sqrt(1+0.5)≈1.22≈22%). In some embodiments, the value α may be selected to be less than 0.33, which limits the maximum thickness to about 0.41 millimeters (using the example materials and values above). In such embodiments, the position walk contribution from the interface corresponds to approximately a 15% increase in position resolution. Accordingly, the various embodiments of spectrally selective crosstalk rejection layers can significantly reduce external crosstalk photons with minimal degradation or impact to interface position resolution.

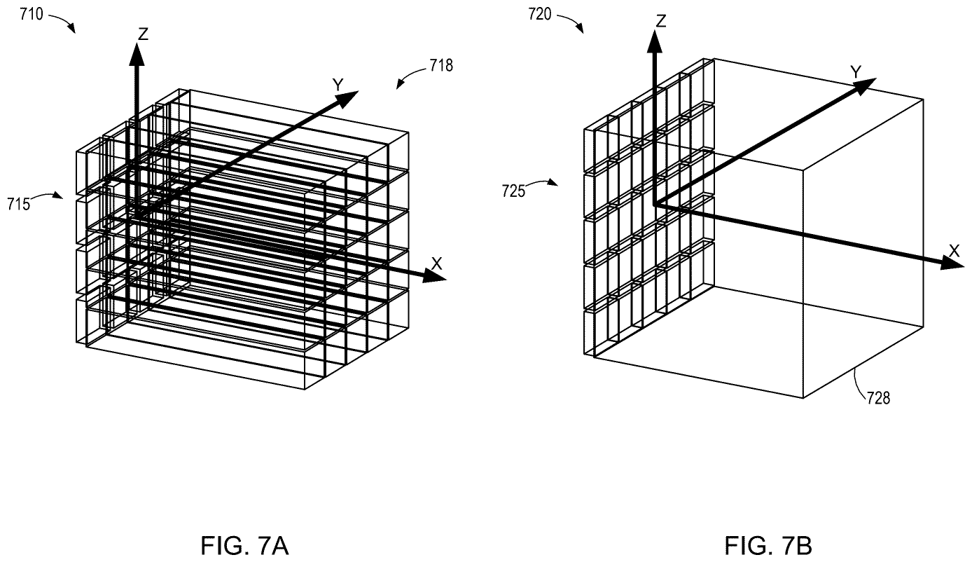

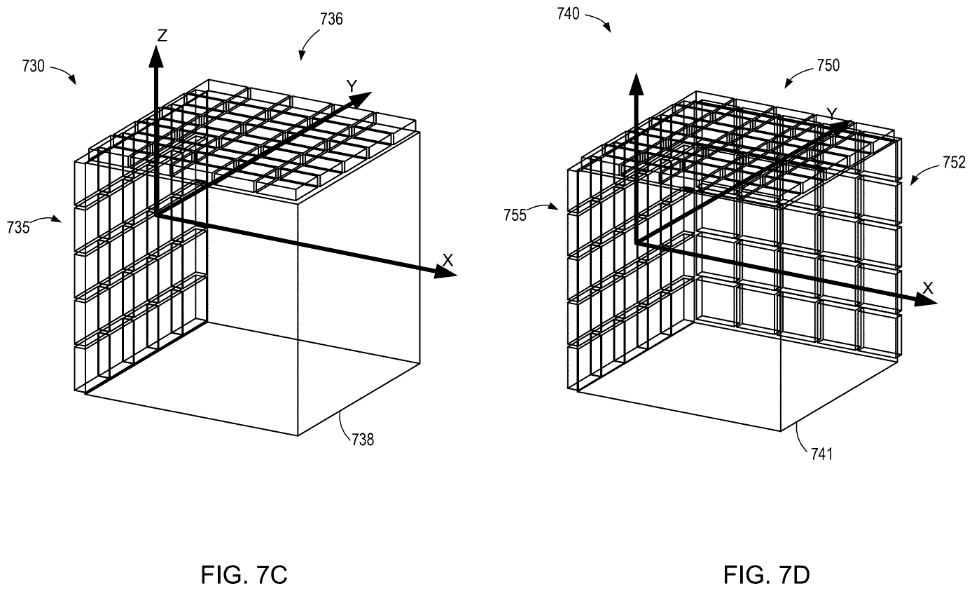

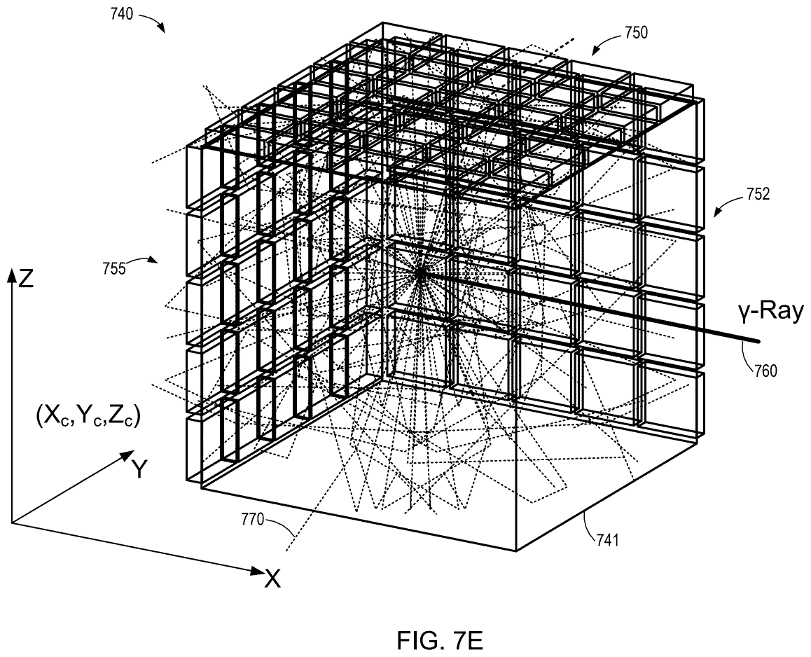

FIG. 7A illustrates high-aspect-ratio pixelated scintillation crystals 718 in a 5×5 array coupled to a 4×4 SiPM array 715, according to one embodiment. As illustrated, the example scintillator detector 710 includes a photodetector array 715 positioned on the scintillation crystals 718 in an end-readout configuration. According to various embodiments, a spectrally selective crosstalk rejection layer (not shown) may be positioned within an optical detection path of the SiPMs 715. The spectrally selective crosstalk rejection layer may be substantially transparent to the scintillation photons generated within the high-aspect-ratio pixelated scintillation crystals 718, but configured to absorb external crosstalk photons from other SiPMs within the 4×4 SiPM array 715 and/or external crosstalk photons from SiPMs of other (e.g., adjacent) scintillator detectors.

FIG. 7B illustrates an example scintillator detector 720 with a monolithic scintillation crystal 728 and a photodetector array 725 positioned on face 5. The scintillator detector 720 includes a 5×5 array of SiPMs 725 in a one-sided end-readout configuration. According to various embodiments, a spectrally selective crosstalk rejection layer (not shown) may be positioned within an optical detection path of the SiPMs 725. The spectrally selective crosstalk rejection layer may be substantially transparent to the scintillation photons generated within the monolithic scintillation crystal 728 while absorbing external crosstalk photons from other SiPMs within the 5×5 array of SiPMs 725 and/or external crosstalk photons from SiPMs of other (e.g., adjacent) scintillator detectors.

FIG. 7C illustrates an example scintillator detector 730 with a monolithic scintillation crystal 738 and a first photodetector array 735 disposed on the end-face (face 5) and a second photodetector array 736 disposed on a lateral face (face 0). Again, spectrally selective crosstalk rejection layers (not shown) may be disposed within the optical detection paths of the SiPMs 735 and the SiPMs 736. The spectrally selective crosstalk rejection layers may be substantially transparent to the scintillation photons but prevent or reduce detection of external crosstalk photons. As described herein, the spectrally selective crosstalk rejection layers may specifically prevent external crosstalk photons from the SiPMs 735 from being detected by the SiPMs 736, and vice versa.

The scintillation photons originating within the monolithic scintillation crystal 738 (i.e., resulting from a scintillation event of received gamma radiation) are detected by the SiPMs 735 and the SiPMs 736 after passing through only one layer of the selective crosstalk rejection layer. In contrast, external crosstalk photons first exit the emitting face of an SiPM (e.g., one of SiPMs 735) and pass through a first spectrally selective crosstalk rejection layer. Before being detected by another SiPM (e.g., one of SiPMs 736), the external crosstalk photons pass through a second spectrally selective crosstalk rejection layer. Each spectrally selective crosstalk rejection layer absorbs or attenuates the external crosstalk photons, reducing the overall noise detected by the SiPMs 735 and 736.

The selective crosstalk rejection layer has negligible or relatively small attenuation at the wavelengths corresponding to the scintillation photons. Accordingly, the single-pass transmission remains high and the added haze is minimal. The asymmetric pass count increases selectivity, such that cross-face crosstalk is strongly attenuated by two passes through the selective crosstalk rejection layer. Meanwhile, the information-bearing scintillation photons only pass through the selective crosstalk rejection layer once and are preserved.

FIG. 7D illustrates an example scintillator detector 740 comprising a monolithic scintillation crystal 741 with three separate photodetector arrays 750, 752, and 755 positioned on faces 0, 2, and 5, respectively, of the scintillation crystal 741 according to one embodiment. Other configurations of multi-sided readout are possible. Faces not covered with SiPM arrays may be covered with reflective material, such as a Teflon film and/or an enhanced specular reflector film. The faces of the scintillation crystal 741 may be polished, and optical coupling material may be used at the interface between the SiPMs 750, 752, and 755 and the scintillation crystal 741.

In a three-face configuration (e.g., with SiPMs on faces 0, 2, and 5), each SiPM array may feed a dedicated timing channel that determines per-face timing information (e.g., using a leading-edge discriminator). A time correction module may receive the estimated interaction location (x,y,z) from the position module and apply calibrated corrections to each face. The adjusted timing signals may be used for coincidence processing and/or depth-of-interaction-aware time-walk compensation.

FIG. 7E illustrates an example scintillator detector 740 comprising a monolithic scintillation crystal 741 with three separate photodetector arrays 750, 752, and 755 positioned on faces 0, 2, and 5, respectively, of the scintillation crystal 741 according to one embodiment. A gamma-ray 760 enters the monolithic scintillation crystal 741 and is scintillated as a plurality of isotropically emitted photons, represented by dashed lines 770. Simulation techniques, such as Monte Carlo simulations, can be used to model scintillation, photon emissions, specular reflection, diffusion, and detection. In some examples, each of the photodetector arrays 750, 752, and 755 comprises a plurality of SiPMs (e.g., a 2×2, 3×3, 4×4, or 5×5 array of SiPMs) or other photodetectors. For example, rectangular SiPMs with length and width dimensions between approximately 2 millimeters and 8 millimeters may be used.

In some embodiments, the monolithic scintillation crystal 741 is a cuboid with equal length, width, and height dimensions of about 20 millimeters. The photodetector array 755 on face 5 of the monolithic scintillation crystal 741 may be, for example, a 5×5 array of 4 mm×4 mm SiPMs, as illustrated. In a different embodiment, the scintillator detector 740 may include a 20 mm×20 mm×20 mm monolithic scintillation crystal 741 with photodetector arrays formed as an 8×8 array of 2 mm×2 mm SiPMs or a 6×6 array of 3 mm×3 mm SiPMs, which may be equispaced upon each detection face of the monolithic scintillation crystal 741. Any face that is not a detection face (e.g., a non-detection face that does not have a photodetector array thereon) and/or any surface area of a detection face that is not covered by a SiPM may be covered by reflective material.

The amount of light generated by a gamma-ray-induced scintillation event varies according to a Gaussian distribution, with the mean amount of light dependent on the energy of the incident gamma ray. However, the variation in the amount of light produced is sufficiently small that, for a given gamma-ray energy, the total light output for events remains approximately constant. Moreover, changes to scintillator geometry and light collection configurations do not affect this per-event scintillation light output. In conventional PET detectors, distributing the scintillation light across a larger number of photodetectors, rather than concentrating it on fewer photodetectors, is typically associated with a degradation in timing performance. Detector timing uncertainty is influenced by various factors, including scintillator rise (τᵣᵢₛₑ) and decay times (τ_{decay}), the number of detected photons (N), single photon rise time of the photodetector (τₛₑᵣ), dark count rate (DCR), and an uncorrelated noise component (σᵤ). The relationship between these factors and the coincidence timing resolution (CTR) can be expressed as follows:

$$CTR ∝ \sqrt{\frac{{τ}_{decay} {τ}_{rise}}{N} + \frac{{τ}_{ser} DCR}{{N}^{2}} + {σ}_{u}^{2}}$$

In this equation, the first term represents the uncertainty from the scintillator, while the second term accounts for the photodetector's signal-to-noise ratio and photodetector noise. Both terms are signal-correlated, as they depend on N. The third term, representing uncorrelated noise, is independent of the signal strength. When multiple photodetectors are employed, the number of detected photons (N) is distributed across these detectors rather than concentrated on a single photodetector. If K photodetectors each read out the scintillation light, the CTR equation becomes:

$$CTR ∝ \sqrt{\frac{{τ}_{decay} {τ}_{rise}}{N} + K * \frac{{τ}_{ser} DCR}{{N}^{2}} + \frac{1}{K} * {σ}_{u}^{2}}$$

In this case, the first term, associated with the scintillator, remains unaffected by the number of detectors. The second term, which dominates the CTR, scales with K, indicating that increasing the number of detectors amplifies the timing uncertainty due to the detector dark count rate and rise time. Thus, dividing the light signal across more photodetectors reduces the signal-to-noise ratio. The third term, representing uncorrelated noise, such as timing jitter from a time-to-digital converter (TDC), decreases inversely with K.

In PET detectors utilizing scintillators like LYSO or LSO and low-noise TDCs, the second term generally dominates the CTR. Therefore, adding more detectors while maintaining the same number of detected photons typically results in a deterioration of timing performance, thereby reducing temporal spatial resolution. For this reason, conventional detector designs use high aspect ratio scintillators with a small area in contact with the photodetector, along with reflective material on the sides, to concentrate all scintillation light onto a small detection area. This configuration ensures that the light is captured by a minimal number of photodetectors, maximizing the signal-to-noise ratio and optimizing the timing resolution equation.

In contrast, in a monolithic scintillator, the light is spread over a larger detection area, which dilutes the light signal across multiple detectors. Traditional monolithic designs generally lead to poorer timing performance compared to high-aspect-ratio scintillator detector geometries because the wider spread of light reduces the concentration of detected photons per photodetector, thereby increasing timing uncertainty. Moreover, incorporating more photodetectors increases the cost and complexity of the detector and readout system. As a result, many traditional PET detectors employ high-aspect-ratio scintillators wrapped in reflective materials to focus scintillation light onto a small number of detectors, thereby minimizing light dispersion and improving timing resolution.

The preceding analysis assumes that the single photon rise time (τₛₑᵣ) remains constant with the addition of more or larger photodetectors. However, this assumption does not hold for most types of photodetectors. In the case of SiPMs, each detector consists of numerous microcells, where each microcell comprises an avalanche photodiode (APD), and in this configuration, it is often termed a single-photon avalanche photodiode (SPAD), and a quenching resistor. These microcells are connected in parallel, and their combined outputs produce the total current of the photodetector. When signals from multiple microcells or discrete SiPMs are combined, the effective τₛₑᵣ increases, leading to a further degradation in CTR.

This poses an additional challenge in detector configurations that utilize a larger number of photodetectors. As the number of SiPMs increases, the effective τₛₑᵣ is further degraded due to the combination of signals and parasitics, which raises the overall capacitance and slows the system's response time. A small number of SiPMs, or even a single SiPM, may be coupled to one end of a high-aspect-ratio scintillator in an attempt to improve or even optimize the photon rise time (τₛₑᵣ). In such a configuration, all the scintillation light is concentrated onto a single SiPM, preventing τₛₑᵣ degradation, such as the τₛₑᵣ degradation that can occur when multiple SiPMs are combined. Existing literature widely regards a “one-to-one” configuration, where a high-aspect-ratio scintillator is paired with a single SiPM, as the optimal solution for minimizing the negative effects of increased capacitance and maintaining superior timing performance.

Monolithic detector designs spread scintillation light over a larger area than pixelated scintillator designs. Monolithic detector designs may use multiple SiPMs to cover the detection area. For example, the light may be distributed across many SiPMs (or another type of photodetector array), leading to a photon rise time τₛₑᵣ degradation as signals from multiple detectors are combined with increasing overall capacitance. As a result, traditional monolithic geometries suffer from poorer timing performance due to the increased capacitance and τₛₑᵣ degradation, making it difficult to achieve the same timing precision as in high-aspect-ratio scintillator-SiPM pairings.

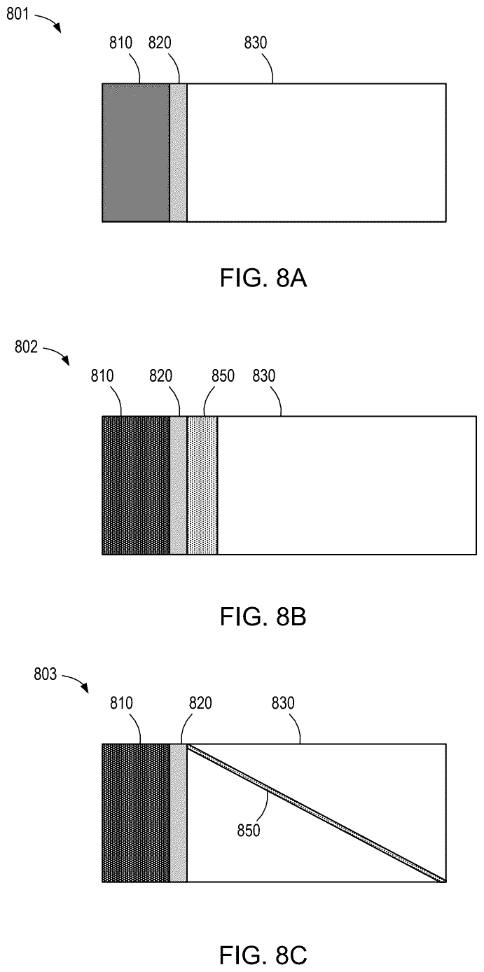

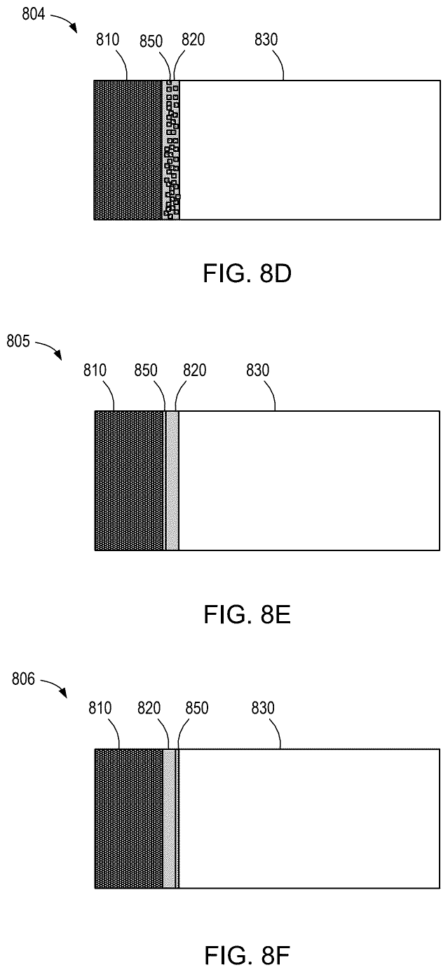

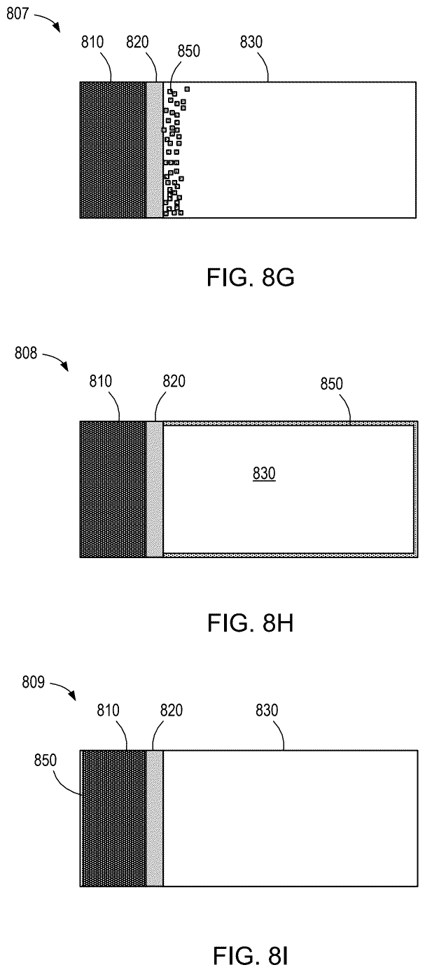

FIGS. 8A-8I illustrates various configurations of spectrally selective crosstalk rejectors used in conjunction with scintillator detectors with SiPMs coupled to scintillation crystals, according to various embodiments. The illustrated examples are simplified to illustrate possible configurations and positioning options for spectrally selective crosstalk rejection films, layers, and embedded materials. The illustrated configurations and placements of spectrally selective crosstalk rejectors may be utilized in conjunction with more complex scintillator detector configurations in which SiPMs are placed on any number of faces of a scintillation crystal (e.g., a monolithic scintillation crystal). For example, the illustrated configurations and placements of spectrally selective crosstalk rejection films, layers, and embedded materials may be utilized in conjunction with any of the previously described embodiments, including those described in conjunction with FIGS. 5A-7E.

FIG. 8A illustrates a scintillator detector 801 with an SiPM 810, an interface layer 820, and a scintillation crystal 830. The interface layer 820 may, for example, be an optical coupling medium. The scintillator detector 801 does not include a spectrally selective crosstalk rejection layer.

FIG. 8B illustrates a scintillator detector 802 with a spectrally selective crosstalk rejector 850 formed as a layer positioned between the interface layer 820 and the scintillation crystal 830. In some embodiments, the spectrally selective crosstalk rejection layer is applied directly to the surface of the scintillation crystal 830 and/or the interface layer 820 (e.g., an optical coupling medium). As in other embodiments, the spectrally selective crosstalk rejection layer 850 absorbs longer-wavelength photons (e.g., external crosstalk photons) while being substantially transparent to shorter-wavelength photons (e.g., scintillation photons).

FIG. 8C illustrates an example of a scintillator detector 803 in which the spectrally selective crosstalk rejector 850 is positioned at an angle relative to the SiPM 810 and interface layer 820 within the scintillation crystal 830.

FIG. 8D illustrates an example of a scintillator detector 804 in which the spectrally selective crosstalk rejector 850 comprises dyes or pigments mixed into an optical coupling medium (e.g., of the interface layer 820). In some embodiments, the spectrally selective crosstalk rejector may comprise rejection materials embedded within the interface layer 820, a cover or layer of the SiPM 810, and/or within another layer of an optical stack (e.g., as part of a multi-layer optical stack) between the SiPM 810 and the scintillation crystal 830. In some embodiments, a spectrally selective crosstalk rejection layer is integrated within a multi-layer optical

FIG. 8E illustrates an example of a scintillator detector 805 with a thin spectrally selective crosstalk rejection layer 850 between the SiPM 810 and the interface layer 820. The thin spectrally selective crosstalk rejection layer 850 may be a self-supporting film, an adhesive film, or a thin coating. In some embodiments, the thin spectrally selective crosstalk rejection layer 850 may be applied to (or integrated into) the SiPM 810 as a thin optical rejection layer on the photodetector.

FIG. 8F illustrates a scintillator detector 806 with a spectrally selective crosstalk rejector 850 formed as a layer positioned between the interface layer 820 and the scintillation crystal 830.

FIG. 8G illustrates a scintillator detector 807 in which spectrally selective crosstalk rejection material 850 is embedded within the scintillation crystal 830 near the interface between the scintillation crystal 830 and the interface layer 820.

FIG. 8H illustrates a scintillator detector 808 in which the spectrally selective crosstalk rejection layer 850 is applied around the external faces of the scintillation crystal 830 that do not have an SiPM 810 (or other photodetectors) thereon.

FIG. 8I illustrates a scintillator detector 809 in which the spectrally selective crosstalk rejection layer 850 is positioned on an external-facing side of an SiPM 810 or other photodetector. Any combination of the illustrated configurations and relative positions of spectrally selective crosstalk rejectors 850, including layers, films, coatings, embedded materials, and the like, may be combined in a given embodiment.

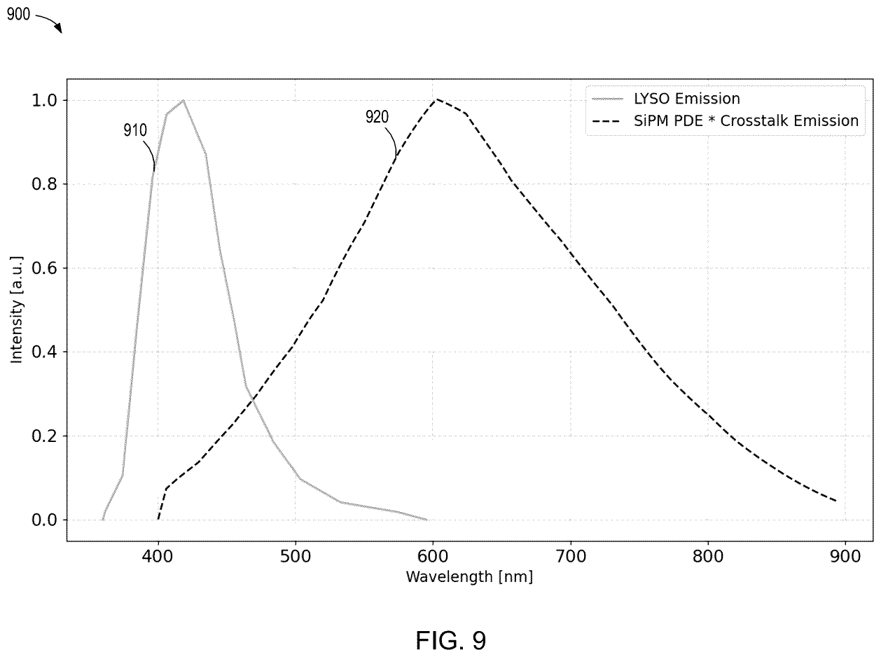

FIG. 9 illustrates a graph 900 of lutetium-yttrium oxyorthosilicate (LYSO) emission spectrum 910 and the product of SiPM photodetection efficiency (PDE) and the SiPM external crosstalk emission spectra 920, according to one embodiment. As illustrated, the LYSO emission spectrum 910 peaks at wavelengths of approximately 375 nanometers to 500 nanometers, which corresponds to the desired wavelengths that are efficiently transmitted through the optical system to ensure accurate radiation detection. The illustrated product of the photodetection efficiency (PDE) of an example SiPM and the spectrum of its crosstalk emission 920, depicts the wavelengths at which optical crosstalk photons are detected. Specific devices may have different spectrums. Accordingly, spectrally selective crosstalk rejectors may be configured or tuned for use with specific combinations of scintillators and SiPM devices, such that the scintillation photon emissions (e.g., LYSO emissions 910) pass through without significant absorptions, while the external crosstalk photons (e.g., crosstalk emission 920) are substantially absorbed or otherwise filtered.

As illustrated, the effective crosstalk spectrum 920 is most intense between approximately 550 nanometers and 900 nanometers, overlapping significantly with the longer wavelength end of the SiPM sensitivity spectrum (e.g., including some infrared wavelengths), thereby causing unwanted noise in the detection system. As described herein, a selective optical crosstalk rejection layer can be substantially transparent to the LYSO emission 910 while rejecting most of the longer-wavelength crosstalk photons 920, thereby reducing dark count rates and improving the overall signal fidelity of the radiation detection system (e.g., increasing the CTR, energy resolution, and/or position resolution).

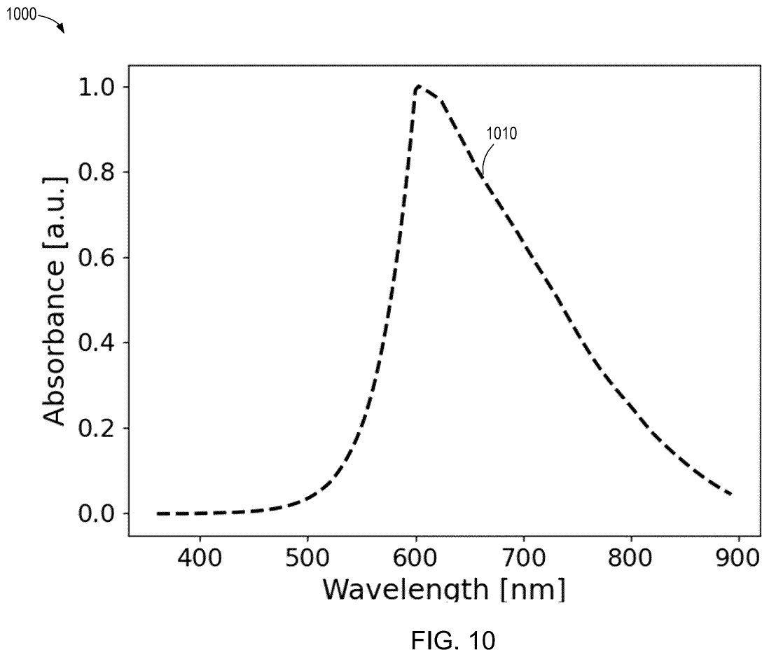

FIG. 10 illustrates a graph 1000 of an example target absorption spectrum 1010, according to one embodiment. The absorption spectrum 1010 of the target rejection material shows high transparency in the range of approximately 375 nanometers to 500 nanometers, which corresponds to the primary emission wavelengths of the scintillation crystal. The target rejection material has a high absorption at wavelengths longer than approximately 500 nanometers (e.g., to approximately 900 nanometers) with a peak around 600 nanometers. The target transmission/absorption profile attenuates optical crosstalk photons emitted by the silicon photomultiplier (SiPM), while allowing the desired scintillation photons to reach the SiPM with minimal loss. Additionally, the refractive index of the rejecting material may be matched to the host material (e.g., to the scintillator, SiPM, SiPM cover, etc.) to minimize optical scattering. For example, a spectrally selective crosstalk rejection layer thin film may have a refractive index between 1.3 and 1.6.

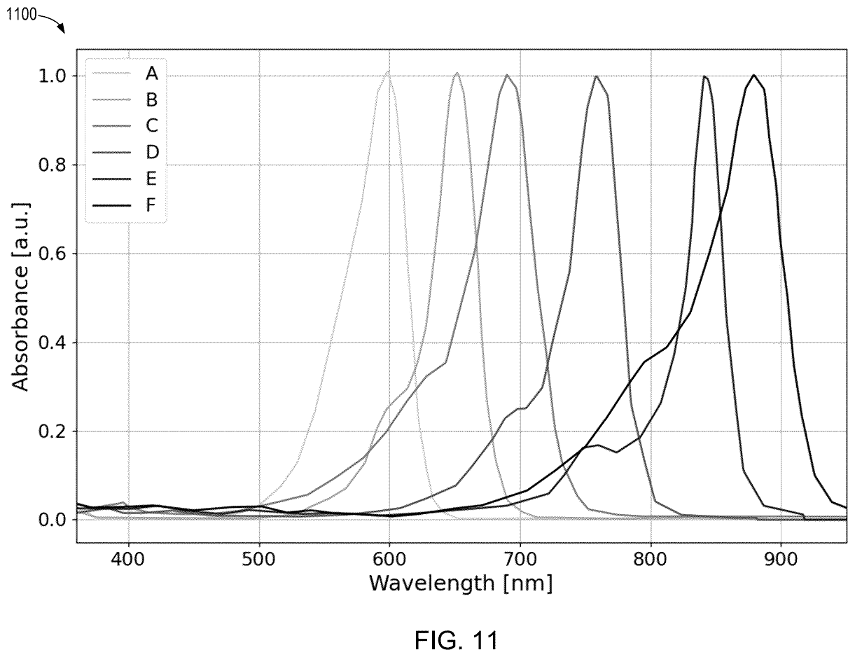

FIG. 11 illustrates a graph 1100 with examples of narrow-band absorption profiles for several optical dyes suitable for use in spectrally selective crosstalk rejectors, according to various embodiments. The absorption bands for dyes “A”, “B”, “C, “D”, “E”, and “F” are depicted. In this example, the dyes are polymethine dyes. Multiple dyes may be combined to approximate a target rejection spectrum over a broader wavelength range by superposing their individual absorption bands. In preferred embodiments, each dye is selected to exhibit negligible absorption across the principal emission band of a scintillation crystal to avoid attenuating scintillation photons. The dyes selected may exhibit low self-fluorescence and have good photochemical stability in a chosen polymer matrix. In some examples, various dyes are combined simultaneously in a matrix carrier material, such as PMMA solvent. The resulting coating mixture is applied to the surface of the SiPM and/or scintillation crystal. In other embodiments, each dye may be applied as a coating or film separately and sequentially, such that an optical stack includes multiple layers of different dyes stacked on top of each other to achieve a target absorption response.

The combination of solvents, carrier matrices, dyes, pigments, and the like are selected for sequential layering or combining to achieve a target rejection (e.g., absorption) spectrum that corresponds to the optical crosstalk emission spectrum of the specific SiPM and photo detection efficiency (as discussed in conjunction with FIG. 9). Suitable dyes include, without limitation, polymethine dyes, which comprise an extended conjugated π-electron system characterized by an odd-numbered sequence of alternating single and double bonds between methine (—CH═) groups, optionally terminating in electron-donating and electron-withdrawing substituents that tune spectral properties. Different dyes exhibit different solubilities in suitable coating matrix materials (e.g., PMMA in anisole). Direct addition of a sparingly soluble dye into the polymer solution can lead to limited loading, aggregation, and non-uniform dispersion in the dried film.

To address this, an intermediate solvent may be utilized that (i) dissolves the dye(s) at useful concentrations and (ii) is miscible with, and non-antisolvent to, the polymer (or matrix material) casting solution. Exemplary intermediate solvents include, but are not limited to, acetone, methanol, dimethyl sulfoxide (DMSO), dimethylformamide (DMF), ethanol, N-methyl-2-pyrrolidone (NMP), and methyl-ethyl-ketone (MEK, 2-butanone).

A specific, non-limiting example formulation includes 0.06 g of dye A and 0.08 g of dye B that are dissolved in 5 mL MEK, and 0.05 g of each dye C, D, E, and F dissolved in 1 mL acetone. The MEK and acetone dye solutions are combined and then blended with 5 mL of PMMA A9 (e.g., PMMA in anisole) to yield a homogeneous rejection solution engineered to be substantially transparent within a given scintillator emission band (e.g., LYSO) and absorptive across the targeted crosstalk wavelengths. Use of the intermediate solvent enables higher effective dye loading without precipitation, promotes molecular-level dispersion in the polymer matrix, and suppresses agglomeration during drying. The resulting spectrally selective crosstalk rejection material achieves a higher optical density even in very thin films.

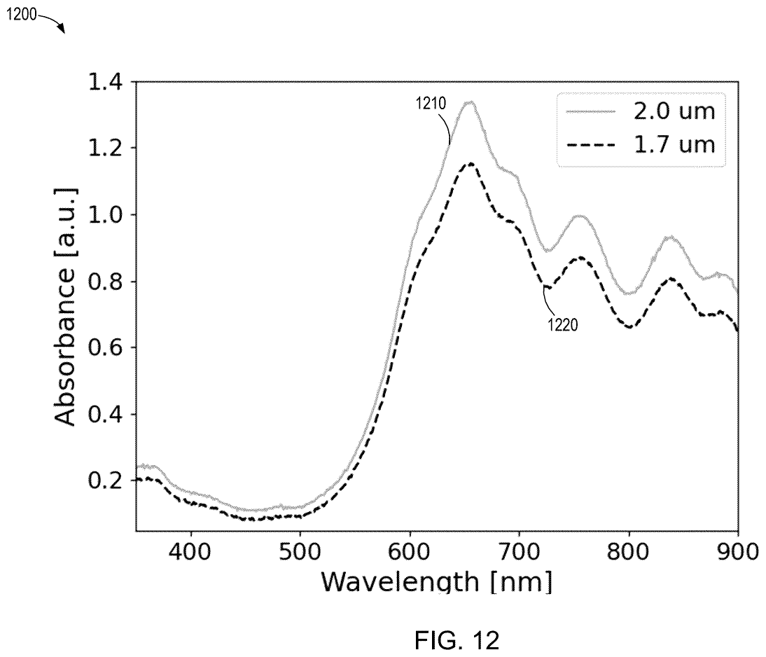

FIG. 12 illustrates a graph 1200 of a rejection spectrum for the example spectrally selective crosstalk rejection material, according to one embodiment. As illustrated, with a thickness of only two micrometers (plot 1210) or even only 1.7 micrometers (plot 1220), a high absorbance of 1.3 can be obtained at 650 nanometers, with good absorbance between 550 nanometers and 900 nanometers (the wavelengths of external crosstalk photons). Thus, the intermediate solvent enables a high loading of the dyes to achieve a spectrally selective crosstalk rejection material that is substantially transparent at wavelengths 400-500 nanometers (e.g., the LYSO emission window) and substantially absorbing at wavelengths between 550 nanometers and 900 nanometers, despite having micrometer thickness.

Examples of suitable matrix materials include, but are not limited to poly(methyl methacrylate) (PMMA) and other acrylics (e.g., poly(ethyl methacrylate) (PEMA), poly(butyl methacrylate) (PBMA)), styrenics (e.g., polystyrene (PS), styrene-acrylonitrile copolymer (SAN)), and cyclic olefin copolymer (COC), and cyclic olefin polymer (COP), as well as other optically clear resins that yield uniform, low-scatter, low-fluorescence coatings with a refractive index in a practical range for optical coupling. In various embodiments, PMMA is utilized because, after solvent evaporation, it forms a high-transparency, conformal layer with a refractive index of about 1.5, which is closely matched to common optical coupling media and many dyes. Matching refractive indices limits or eliminates Fresnel reflections and parasitic scatter. The thickness of the spectrally selective crosstalk rejection material, film, coating, layer, etc., can be set reproducibly by spin speed and solids content, enabling consistent coatings over large areas and across multiple scintillator or SiPM surfaces.

For example, a small droplet of the spectrally selective crosstalk rejection solution may be deposited onto the detection surface of an SiPM, an opposing back surface of an SiPM, and/or one or more surfaces of a scintillation crystal. With the droplet of the spectrally selective crosstalk rejection solution deposited, the surface can be spun at speeds between 500 and 4000 rpm for several seconds to achieve a target thickness (e.g., 20-60 seconds) and baked or otherwise heated to form a uniform thin absorption film or coating (e.g., baked at temperatures ranging from 50 to 120° C.).

In another specific example, the dye material is a mixture of polymethine dyes in acetone, methanol, or Ethyl methyl ketone, and the carrier material is PMMA in anisole. In such an example, the polymethine dyes and PMMA in anisole are spin-coated onto either the SiPM or the scintillation crystal by dropping a small droplet on the surface, spinning at 500 rpm for 5-10 seconds, spinning at 1000-4000 rpm for 30-60 seconds, and then heating at 50 to 150° C. for 1-5 minutes.

In these embodiments, the polymer functions as the dye carrier (matrix material), solubilizing and conformally encapsulating the dye molecules. Upon drying/curing, the matrix immobilizes the dyes, reduces aggregation and leaching, and limits oxygen/moisture ingress, thereby improving long-term photochemical and thermal stability under PET operating conditions. Optional crosslinkers, barrier over-coats, and stabilizer additives (e.g., antioxidants) may be incorporated to further enhance durability without departing from the scope of the disclosure.

In further embodiments, the rejection solution is deposited by non-spin techniques chosen for large-area coverage, patterned application, or multi-face coating of a scintillator block or SiPM package. In some embodiments, slot-die coating may be utilized with a closed-aperture head to deliver a uniform wet film with a thickness governed by volumetric flow, head speed, coating gap, and solution solids content. Upon drying, the film yields the targeted optical density while maintaining low scatter. In some embodiments, doctor-blade (bar or Meyer-rod) coating may be used with controlled thickness setting via blade gap and traverse speed. In some embodiments, printing methods such as inkjet, aerosol-jet, screen, gravure, or flexographic printing may be used for patterned or selective deposition over photosensor arrays, enabling registration to bond pads or passivation windows. In some cases, a shadow mask or temporary resist may be used to define the coated regions with subsequent lift-off. In some embodiments, dip-coating may be used to treat multiple faces sequentially, with withdrawal speed and solution rheology controlling the wet film thickness and uniformity. In some embodiments, spray or curtain coating may be used to achieve conformal coverage over complex geometries. In some embodiments, a spectrally selective crosstalk rejection layer thin film may be conformally applied to a curved surface.

In some embodiments, an intermediate-solvent strategy may be utilized, where each dye is first dissolved in a solvent that is highly soluble for the dye and miscible with, and at the concentration used, a non-antisolvent to the polymer matrix material, followed by blending into the polymer casting solution to obtain a stable rejection solution. The coated surfaces may then be dried or baked to remove residual solvent and densify the film, producing optically clear, uniform, and highly absorptive layers that remain substantially transparent across the scintillation photon emission band. Examples of suitable absorbing dyes include but are not limited to (i) Hepta/hexamethine cyanines, hemicyanines, and merocyanines (polymethines), (ii) Squaraines and croconaines, (iii) Naphthalocyanines and metallophthalocyanines, (iv) Aza-BODIPY and red/NIR-tuned BODIPY derivatives, (v) Transition-metal dithiolene complexes, (vi) engineered perylene/naphthalene diimides, and (vii) Metallonaphthalocyanines/metallophthalocyanines.

In some embodiments, a spectrally selective crosstalk rejector is formed as a conformal, non-self-supporting film directly on the scintillation crystal and/or the SiPM by depositing a liquid coating composition (solution or dispersion) that wets and levels the surface, thereby displacing or dispelling air and eliminating interfacial gaps (e.g., air gaps). In various example, the spectrally selective crosstalk rejector may include (i) a composition with a film-forming polymer matrix (e.g., PMMA, PEMA, PBMA, PS, SAN, COC/COP), (ii) one or more absorbers selected to be substantially non-absorbing at wavelengths between approximately 350 and 500 nanometers and absorbing at wavelengths between approximately 550 and 900 nanometers (e.g., polymethines, squaraines, naphthalocyanines, aza-BODIPYs, dithiolenes), and (iii) a solvent vehicle comprising a polymer casting solvent (e.g., anisole) and an intermediate solvent (e.g., MEK, acetone, methanol, ethanol, NMP, DMSO) that dissolves the absorber(s) while being miscible with and non-antisolvent to the polymer.

Representative solids may be ~0.1-10 wt %. In one example process, the target surface is cleaned and optionally surface-activated (e.g., oxygen plasma) and/or primed with an adhesion promoter (e.g., methacrylate-silane). A measured volume of the coating solution is dispensed and spin-coated (e.g., ~500 rpm for 5-10 seconds then ~1,000-4,000 rpm for 30-60 seconds) or slot-die/doctor-blade/inkjet/spray/dip/curtain-coated to a wet film that dries to for a film with a thickness of for example 300 μm, 50 μm, 10 μm, or even less than 2 μm in many embodiments. A brief bake (e.g., 50-150° C. for 1-5 min) removes residual solvent and densifies the film. In some embodiments, a post-cure procedure (thermal or UV) may be used to crosslink the matrix. In many embodiments, the spectrally selective crosstalk rejection material is a liquid layer that can conform to the micro-topography of the surface, and thus does not require a separate couplant, thereby avoiding air gaps and wedge/cavity effects associated with rigid plates.

In some embodiments, the spectrally selective crosstalk rejection layer may first be deposited on a carrier film (e.g., polyethylene terephthalate (PET), COC, PMMA, fluorinated ethylene propylene (FEP), polytetrafluoroethylene (PTFE), Ethylene Tetrafluoroethylene (ETFE), Per- and polyfluoroalkyl substances (PFAS), polypropylene (PP), polyethylene (PE), some of which may be for example coated with silicone or other release coatings) over a release layer or solvent-activatable tie-coat and then transferred to the scintillator or SiPM by lamination. The donor film carrying the absorber layer may be brought into contact under controlled temperature and pressure (e.g., between 4° and 90° C. and between 0.1 and 0.5 MPa) to achieve intimate optical contact without entrapped air. The carrier may then be peeled or otherwise released, leaving the absorber as a conformal coating or conformal film. In some embodiments, a very thin optical adhesive (e.g., less than 10 μm, with an index of refraction of ~1.3-1.6, and exhibiting low fluorescence) or a solvent-fusion is used to bond the spectrally selective crosstalk rejection layer to the surface of the scintillation crystal or the SiPM.

In various embodiments, the resulting spectrally selective crosstalk rejection films are thinner than 300 micrometers, exhibit low-haze (≤~1% at 450 nanometers), are index-matched (~1.3-1.6), provide optical densities less than 0.20 for wavelengths between 350 and 500 nanometers, and provide optical densities of at least a target value between 0.3 and 1.0 for wavelengths between 550 and 900. Because rejection is achieved predominantly by absorption of photons having wavelengths between 550 and 900 nanometers, passband photons are transmitted with minimal reflection or spread, and no air gap is introduced. In one example embodiment, the spectrally selective crosstalk rejection layer thin film has a thickness of less than 10 micrometers, an optical density of at least 0.5 for optical wavelengths between 600 nanometers and 900 nanometers, and an optical density of less than 0.3 for optical wavelengths between 350 and 500 nanometers.

In some embodiments, the spectrally selective crosstalk rejection layer may be pre-formed on a carrier film by, for example, doctor-blading (knife-over-roll) and subsequently transferred to the surface of the scintillation crystal or SiPM. In one exemplary process, a carrier film (e.g., FEP, PET, COC, or PMMA, 25-125 μm) is first coated with a release layer (e.g., silicone or fluoropolymer peel-release, 50-200 nanometers; or a water-soluble PVA release, 100-500 nanometers). A liquid coating composition is then prepared by (i) dissolving each dye (e.g., polymethine, squaraine, naphthalocyanine, aza-BODIPY, or dithiolene) in an intermediate solvent that is highly soluble for the dye and non-antisolvent to the polymer (e.g., MEK, acetone, methanol, ethanol; optionally, ≤10% NMP or DMSO for difficult dyes), (ii) combining the dye concentrates, and (iii) blending the combined dye concentrates into a polymer casting solution (e.g., PMMA in anisole, “A9”) to reach ~0.1-10 wt % total solids. The mixture may be gently rolled or sonicated to homogeneity and filtered (0.2 μm PTFE) to remove particulates/aggregates.

For doctor-blade coating, the blade gap and line speed may be selected to achieve the target rejection layer thickness. Representative conditions are gap 10-120 μm, speed 0.1-1.0 m s⁻¹, solution viscosity 20-200 cP; side dams or edge-wipes limit edge-bead, subsequent drying/curing in an oven, and optional UV crosslinking. To transfer the absorber film, the coated carrier may be laminated to the surface of the scintillation crystal or SiPM using thermal or solvent-assisted lamination. In one approach, the target face is optionally surface-activated (e.g., 02 plasma) and/or primed with a solvent-activatable tie-coat (e.g., ultrathin PMMA/COC micro-tie or methacrylate-silane) that becomes tacky upon brief exposure to anisole/MEK. The absorber-coated carrier may be brought into contact under 40-90° C., 0.1-0.5 MPa nip pressure, with a center-out rolling action (or vacuum-assist) to exclude air, with a contact time between 5 and 60 seconds.

After adhesion sets (solvent flash-off or cool-down), the carrier may be peeled (peel-release) or the release layer may be dissolved (e.g., water rinse for PVA), leaving a conformal, non-self-supporting absorber film on the device. In some embodiments, a very thin optical adhesive (e.g., with a thickness of less than 10 μm, an index of refraction of ~1.3-1.6, and low fluorescence) may be used, or solvent-fusion bonding may be employed, while maintaining an effective added gap of less than 10 μm. A short post-bake (e.g., at 50-120° C. for 1-5 minutes) may be used to complete densification. This carrier-based process enables uniform, ultrathin films with engineered optical densities for wavelengths between 550 and 900 nanometers and minimal passband loss at wavelengths between 350 and 500 nanometers. Moreover, because the spectrally selective crosstalk rejector layer is formed from liquid and transferred under pressure/activation, no air gap is introduced, and the information-bearing light-spread is preserved.

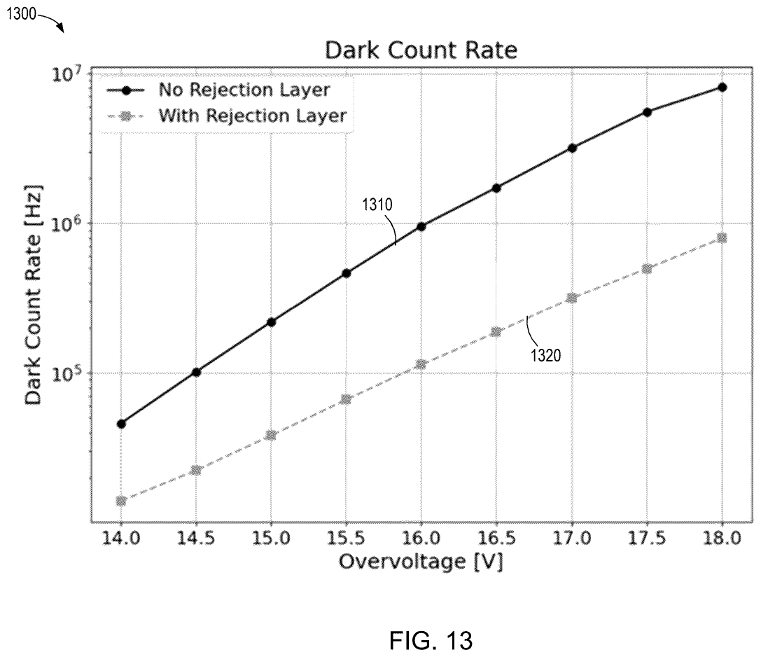

FIG. 13 illustrates a graph 1300 of detected dark count rates (DCR) for an SiPM as a function of overvoltage with a rejection layer 1310 and without a rejection layer 1320, according to one embodiment. The graph 1300 illustrates the detected dark count rates (DCR) for a 20×20×20 mm monolithic LYSO crystal with SiPMs on three orthogonal sides (e.g., as depicted in FIGS. 7D and 7E) and reflective coating on the remaining faces as a function of overvoltage with a crosstalk rejection layer 1310 and without a crosstalk rejection layer 1320 coated on the three orthogonal sides with SiPMs. The graphed values demonstrate that a reduction in DCR by nearly an order of magnitude can be attained by using a crosstalk rejection layer.

The contribution of optical crosstalk to the DCR is smaller at lower overvoltages. For example, at an overvoltage of 14V, the DCR without a rejection layer is 47 kHz and with a rejection layer 14 kHz (i.e., a 3.3× reduction in DCR). At an overvoltage of 18V, the DCR without the rejection layer is 8 MHz while the DCR with the rejection layer is 800 kHz (i.e., a 10× reduction in DCR). Thus, while a crosstalk-rejection layer provides a benefit across the full operating range, its relative effectiveness increases with overvoltage, thereby substantially reducing false triggers and the associated dead time in the radiation-detection system at higher overvoltages.

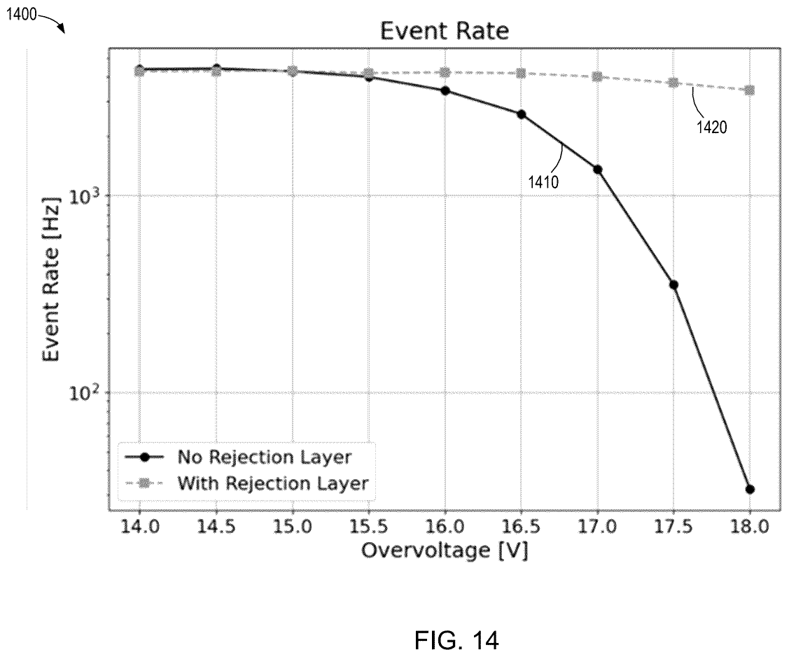

FIG. 14 illustrates a graph 1400 of the number of detected events as a function of overvoltage for an example scintillator detector with SiPMs on three orthogonal sides, according to one embodiment. The graph depicts the number of detected events by the scintillator detector as a function of overvoltage for a 20×20×20 mm LYSO scintillator with SiPMs on three orthogonal sides and a 50 uCi ²²Na radiation source. The number of detected coincidence events with a crosstalk rejection layer 1420 remains relatively stable at overvoltages between 14V and 18V. In contrast, the number of detected coincidence events without a crosstalk rejection layer 1410 decreases exponentially at overvoltages greater than approximately 16V.

The sharp drop in detected coincidence events without the rejection layer at higher overvoltages is due to the optical crosstalk-induced dark counts. Each dark count drives the discriminator/TDC/readout chain into an effective holdoff interval (τ_deadtime) during which no additional events can be detected. As the product DCR*τ_deadtime increases, the window in which coincidence events can be detected decreases. Without a rejection layer, the DCR rises steeply with overvoltage, and the coincidence rate becomes dead-time limited. With a rejection layer, the DCR is significantly reduced, allowing for a sustained high coincidence rate even at elevated overvoltages. In a PET system, this mitigation limits false triggers and associated acquisition deadtime, improving effective count-rate performance without penalizing true-event detection.

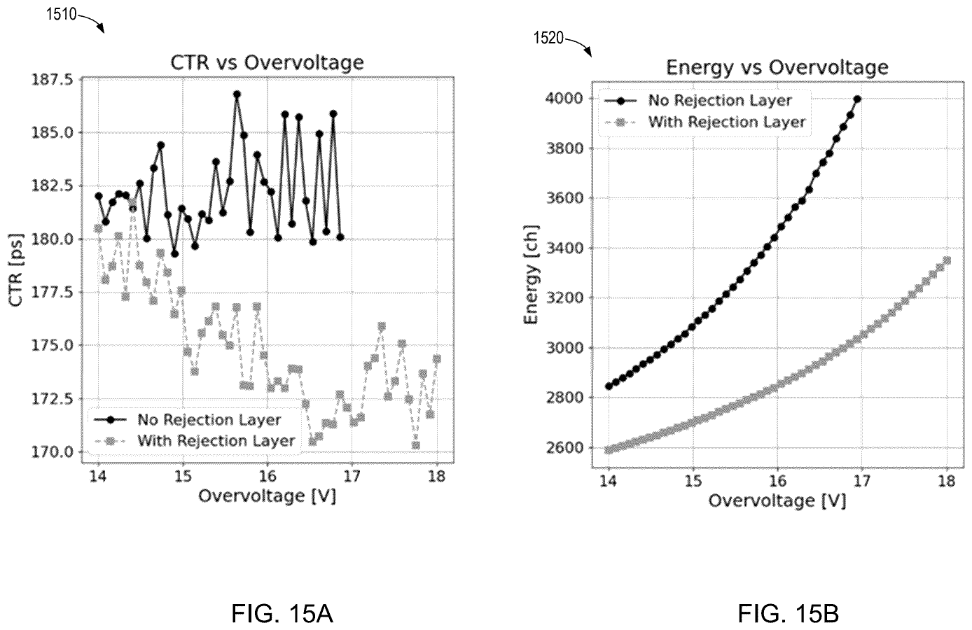

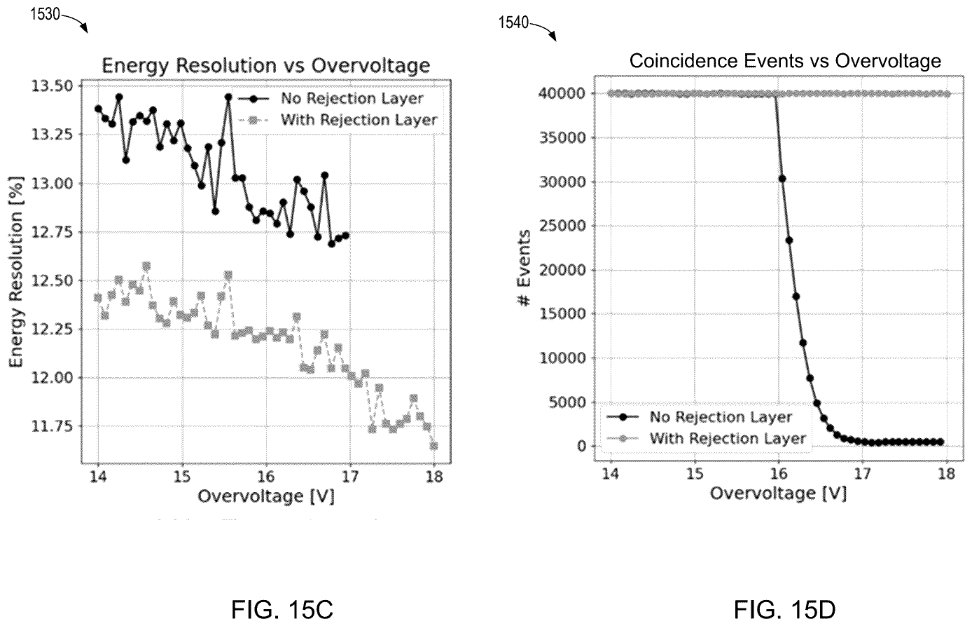

FIG. 15A illustrates a graph 1510 of the coincident time resolution (CTR) with respect to overvoltage values for an example scintillator detector with and without a spectrally selective crosstalk rejector (“rejection layer”), according to one embodiment. The CTR is depicted as a function of overvoltage for a 20×20×20 mm LYSO monolith with SiPM arrays on three mutually orthogonal faces. For each overvoltage setting, 40,000 coincidence events were acquired (capped at a maximum acquisition time of 18 minutes per setting) to ensure comparable statistical confidence across the data points. In the absence of a crosstalk-rejection layer, the CTR remains comparatively flat across ~14-17 V overvoltage, with a minimum of approximately 179.3 ps at 14.9 V. By contrast, when a thin crosstalk rejection layer is applied on the three faces of the monolith with SiPM arrays, the CTR improves across the entire measured range, with the magnitude of improvement increasing at higher overvoltage.

For example, at 16.5 V, the CTR is approximately 170.4 ps with the thin crosstalk rejection layer, representing a significant improvement relative to the best value obtained without the rejection layer. Thus, the crosstalk rejection layer enables SiPM operation at higher overvoltages while preserving high timing resolution, which is useful for applications that rely on precise event timing, such as time-of-flight PET imaging.

FIG. 15B illustrates a graph 1520 of the measured photopeak energy as a function of overvoltage for the example scintillator detector (e.g., configured with three-sided, orthogonal SiPM detection) with and without a spectrally selective crosstalk rejection layer, according to one embodiment. As illustrated, the photopeak measured with the rejection layer is systematically lower than the photopeak measured without the rejection layer across the entire bias range.

FIG. 15C illustrates a graph 1530 of the energy resolution as a function of overvoltage for the example scintillator detector with and without the spectrally selective crosstalk rejection layer, according to one embodiment. As noted with reference to FIG. 15B, the photopeak measured with the rejection layer is systematically lower than the photopeak energy measured without the rejection layer across the entire bias range. However, the graph 1530 shows that the corresponding energy resolution is improved with the use of a rejection layer. The lower measured energy (FIG. 15B) with the rejection layer is attributable to the suppression of external optical crosstalk that, in the no rejection layer configuration, is convolved with the true scintillation signal and artificially inflates the integrated charge. During an avalanche, external crosstalk photons trigger secondary avalanches, and these correlated avalanches fall within the shaping/integration window and are thus summed with the primary signal, which shifts the apparent photopeak upward. The spectrally selective crosstalk rejection layer inhibits propagation of external crosstalk photons, yielding a lowered measured photopeak that more accurately reflects the intrinsic light yield of the crystal-photodetector system.

Thus, the energy resolution (FIG. 15C) improves with the use of a spectrally selective crosstalk rejection layer because suppressing correlated avalanches reduces excess noise factor and event-to-event gain fluctuation. Surprisingly, the rejection layer reduces variance more than it reduces the mean signal amplitude, producing a narrower photopeak (lower FWHM/peak) across the overvoltage range. This improvement in quantitative energy performance complements the timing benefits described above and is advantageous for PET scanner performance for scatter rejection and overall image quality. Furthermore, the energy resolution improves with higher bias voltages, with and without the use of a rejection layer. However, the maximum overvoltage that can be used without a rejection layer is approximately 15.5 V before a significant drop in coincidence rate occurs (where energy resolution is about 13%). In contrast, the maximum overvoltage that can be used with a rejection layer remains stable up to 18V and possibly beyond without a significant drop in event rate (where energy resolution is about 11.7%).

FIG. 15D illustrates a graph 1540 of the number of coincidence events as a function of overvoltage for the example scintillator detector with and without the spectrally selective crosstalk rejection layer, according to one embodiment. The graph 1540 shows the number of coincidence events recorded per overvoltage setting under a fixed acquisition protocol (target of 40,000 coincidence events per setting with an 18-minute timeout). With the use of the spectrally selective crosstalk rejection layer, the system attains the 40,000-event target across the full bias range. In contrast, without the rejection layer, the number of recorded events declines sharply beyond approximately 16 V, approaching zero beyond approximately 17 V as the acquisition repeatedly times out before reaching the 40,000-event threshold. Thus, at elevated overvoltages, scintillator detectors without spectrally selective crosstalk rejection layers become effectively statistics-limited, preventing the collection of sufficient counts for reliable characterization.

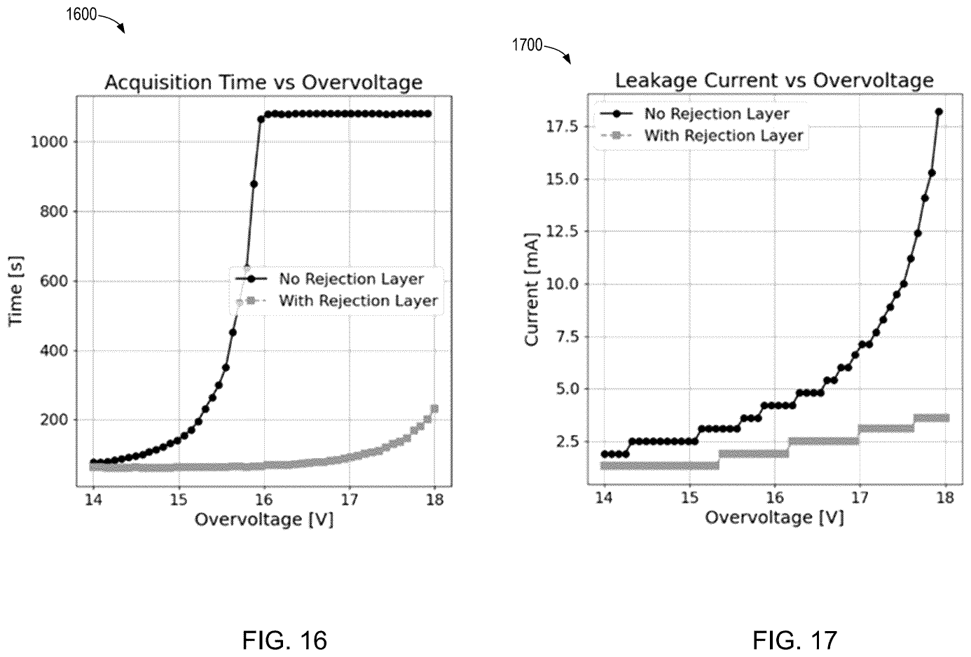

FIG. 16 illustrates a graph 1600 of the acquisition time as a function of overvoltage for the example scintillator detector with and without the spectrally selective crosstalk rejection layer, according to one embodiment. The graph 1600 shows the time required to reach 40,000 coincidence events as a function of overvoltage. The acquisition time for a scintillator detector with a spectrally selective crosstalk rejection layer remains low and increases only marginally above about 16.5 V, consistent with modest dead-time growth. By comparison, for a scintillator detector without a spectrally selective crosstalk rejection layer, the acquisition time increases beginning at approximately 14.25 V and continues to worsen with bias, ultimately reaching the 18-minute cap near ~16 V and failing to complete thereafter.

FIG. 17 illustrates a graph 1700 of the leakage current as a function of overvoltage for the example scintillator detector with and without the spectrally selective crosstalk rejection layer, according to one embodiment. The graph 1700 shows an unexpected benefit of using a spectrally selective crosstalk rejection layer. In this context, leakage current refers to the current drawn from the SiPM high voltage bias supply. In a scintillator detector with a spectrally selective crosstalk rejection layer, the leakage current increases from approximately 1.4 mA at 14 V to about 3.56 mA at 18 V. In stark contrast, in a scintillator detector without a rejection layer, the leakage current is approximately 1.9 mA at 14 V and then rises approximately exponentially to about 18 mA at 18 V.

The reduction in leakage current attained by using a spectrally selective crosstalk rejection layer may be attributable to the suppression of optically induced correlated avalanches (external optical crosstalk) that otherwise elevate both the apparent dark rate and the average avalanche charge drawn per unit time. Lower leakage current reduces power dissipation and self-heating, improving gain stability and mitigating the positive thermal feedback that can drive DCR runaway at high overvoltage. As a result, a scintillator detector with a spectrally selective crosstalk rejection layer remains in a more linear, low-noise operating regime, supporting higher usable bias, better effective count-rate performance (NECR), and more stable timing and photopeak energy in PET systems.

In certain embodiments, a radiation scintillator detector estimates interaction position of the incoming radiation within the scintillator using per-sensor signal amplitudes from the SiPM array(s) on one or more faces (e.g., three, mutually orthogonal faces), employing, for example, Anger logic (energy-weighted centroid), lookup-table/light-response function methods, maximum-likelihood estimators, or learned (e.g., neural-network) regressors implemented in analog and/or digital circuitry. External optical crosstalk, when not mitigated, injects non-informative charge into SiPMs that is not related to the spatial light-sharing pattern of the true scintillation event. This random, position-uncorrelated contribution flattens the position-specific light response distribution, reducing the Fisher information available to any position estimator and resulting in degraded spatial resolution.

By incorporating a thin spectrally selective crosstalk rejection layer near the radiation detector (e.g., SiPM), the position-uncorrelated signals (external crosstalk photons) are suppressed. The signal-to-noise ratio of the position-dependent light distribution needed for accurate localization is increased. Under otherwise identical operating conditions, a representative embodiment exhibits an improvement in transverse and depth of interaction position resolution from approximately 3.2 mm FWHM (in embodiments without a rejection layer) to approximately 2.8 mm FWHM (in embodiments with a rejection layer). Moreover, scintillator detectors with spectrally selective crosstalk rejection layers can be operated at higher overvoltage. Improved intrinsic position resolution is advantageous in PET modules, supporting sharper system point-spread functions and improved lesion detectability.

As demonstrated herein, a micron-scale spectrally selective crosstalk rejection layer suppresses the overvoltage-driven growth of dark count rate and high-voltage leakage current, preserves coincidence count rate to high overvoltages, and improves coincidence timing and energy resolution. For example, DCR reductions of about 3.3× at 14 V and about 10× at 18 V can be attained, accompanied by stabilized leakage current and maintained coincidence rates at elevated bias. Given that thick absorbers do not exhibit improved coincidence time resolution, energy resolution, or position resolution, the results described herein may seem counterintuitive. However, the results demonstrate that thin spectrally selective crosstalk rejection layers, according to the embodiments described herein, provide unexpectedly effective external crosstalk mitigation, together with improved coincidence time resolution, energy resolution, and position resolution. The improvements are particularly notable in the context of scintillator detectors with SiPM on multiple faces, including multi-face monolithic readout configurations.

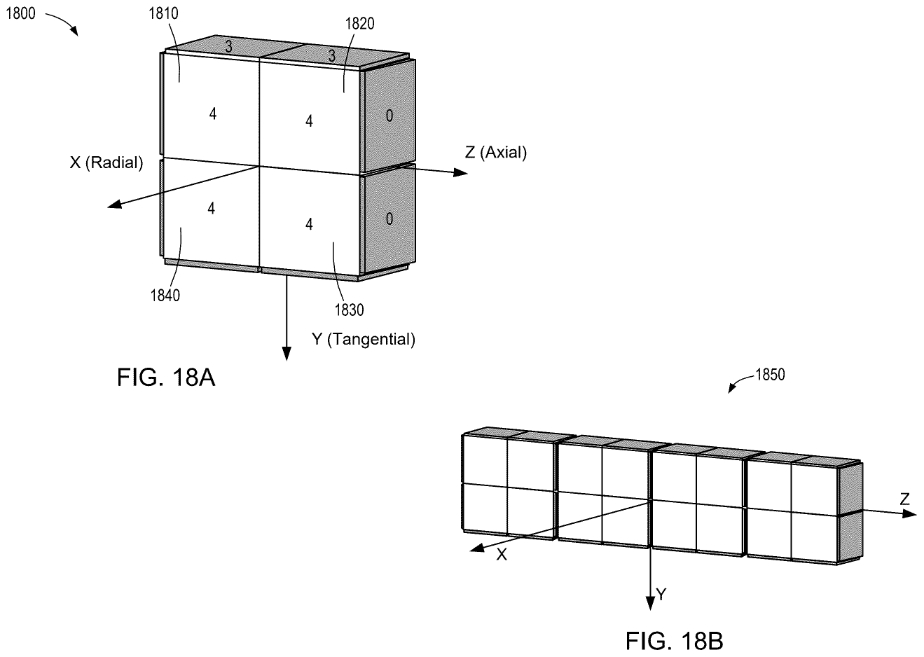

FIG. 18A illustrates an example of a radiation detector module 1800 with four scintillator detectors arranged in a 2×2 array, where each scintillator detector comprises a scintillation crystal 1810, 1820, 1830, and 1840 and multiple photodetector arrays. As illustrated, a photodetector array is positioned on multiple faces of each scintillation crystal 1810, 1820, 1830, and 1840. The photodetector arrays are placed on the lateral faces in a rotated configuration that allows for the inner faces of the scintillation crystals to be in direct contact with one another, separated only by the reflective material on each of the inner faces face. Accordingly, the photodetector arrays on lateral faces of the scintillation crystals in the 2×2 array of scintillator detectors are positioned on faces that are nonadjacent to the other scintillation crystals in the 2×2 array of scintillator detectors. As illustrated, scintillation crystal 1810 includes photodetector arrays on faces 1, 3, and 5 (face 5 is the back side and is not visible). Scintillation crystal 1820 includes photodetector arrays on faces 0, 3, and 5. Scintillation crystal 1830 includes photodetector arrays on faces 0, 2, and 5. Scintillation crystal 1840 includes photodetector arrays on faces 1, 2, and 5.

The radiation detector module 1800 includes a 2×2 array of scintillator detectors in a configuration that utilizes the virtual crystal as detector area. The inner end-faces of the four scintillation crystals (face 4) are configured to be radially oriented into the detector volume (e.g., into a detector ring). Each lateral face of each scintillation crystal with reflective material is in contact with a lateral face of another scintillation crystal with reflective material. The inter-crystal spacing of the scintillation crystals within the detector module 1800 is minimized while still allowing for each scintillation crystal to have a photodetector array on multiple orthogonal faces.

FIG. 18B illustrates a radial sector 1850 with four radiation detector modules (similar to radiation detector module 1800 of FIG. 18A). Each radiation detector module includes four scintillator detectors, where each scintillator detector includes a scintillation crystal with orthogonal photodetector arrays on multiple sides, as described in conjunction with FIG. 18A. In this configuration, the photodetectors occupy the virtual crystal gap between adjacent radiation detector modules. In some embodiments, each radial sector may include more modules. A detector ring may include any number of radial sectors to achieve a target diameter with a relatively close spacing of radial sectors. A PET scanner may include any number of axially aligned detector rings.

The photodetectors of each scintillator detector in the radiation detector module are positioned within a virtual crystal gap between adjacent radiation detector modules of the radial sector 1850. The virtual crystal gap between adjacent radiation detector modules may be configured to have a width or crystal gap spacing corresponding to an integer multiple of a spatial resolution bin size of the radiation detector modules. For example, the virtual crystal gap between adjacent radiation detector modules may be configured to have a width equal to the spatial resolution bin size of the detector module, twice the spatial resolution bin size, three times the spatial resolution bin size, etc.

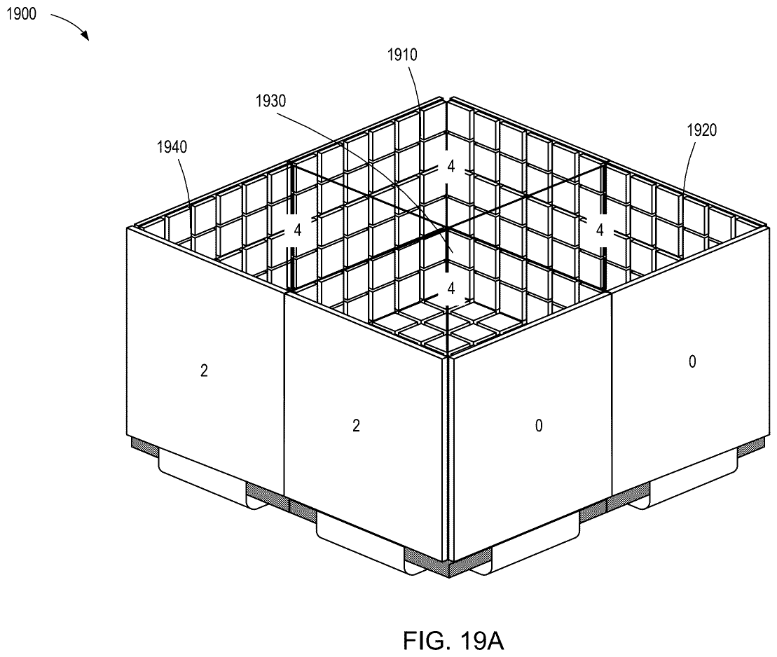

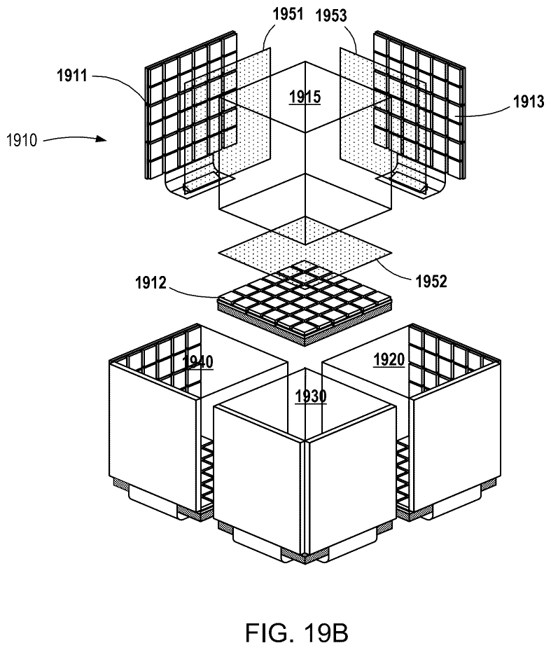

FIG. 19A illustrates an example of a radiation detector module 1900 with four scintillator detectors arranged in a 2×2 array with spectrally selective crosstalk rejection layers between the SiPMs and the faces of the scintillation crystals, according to one embodiment. Each scintillator detector comprises a scintillation crystal 1910, 1920, 1930, and 1940 and multiple photodetector arrays. As illustrated, a photodetector array is positioned on multiple faces of each scintillation crystal 1910, 1920, 1930, and 1940. Specifically, the photodetector arrays are placed on the lateral faces in a rotated configuration that allows for the inner faces of the scintillation crystals to be in direct contact with one another (e.g., separated only by the reflective material on the inner faces. Accordingly, the photodetector arrays on lateral faces of the scintillation crystals in the 2×2 array of scintillator detectors are positioned on faces that are nonadjacent to the other scintillation crystals in the 2×2 array of scintillator detectors.

A spectrally selective crosstalk rejection layer is positioned between the SiPMs and the faces of the scintillation crystals. In the illustrated embodiment, scintillation crystal 1910 includes photodetector arrays and corresponding spectrally selective crosstalk rejection layers on faces 1, 3, and 5 (face 5 is the bottom side and is not visible). Scintillation crystal 1920 includes photodetector arrays and corresponding spectrally selective crosstalk rejection layers on faces 0, 3, and 5. Scintillation crystal 1930 includes photodetector arrays and corresponding spectrally selective crosstalk rejection layers on faces 0, 2, and 5. Scintillation crystal 1940 includes photodetector arrays and corresponding spectrally selective crosstalk rejection layers on faces 1, 2, and 5.

FIG. 19B illustrates the example radiation detector module of FIG. 19A with the scintillator detector 1910 shown in an exploded view, according to one embodiment. As illustrated, the scintillator detector 1910 includes a scintillation crystal 1915. Spectrally selective crosstalk rejection layers 1951, 1952, and 1953 are positioned between the SiPMs 1911, 1912, and 1913 and faces 1, 3, and 5 of the scintillation crystal.

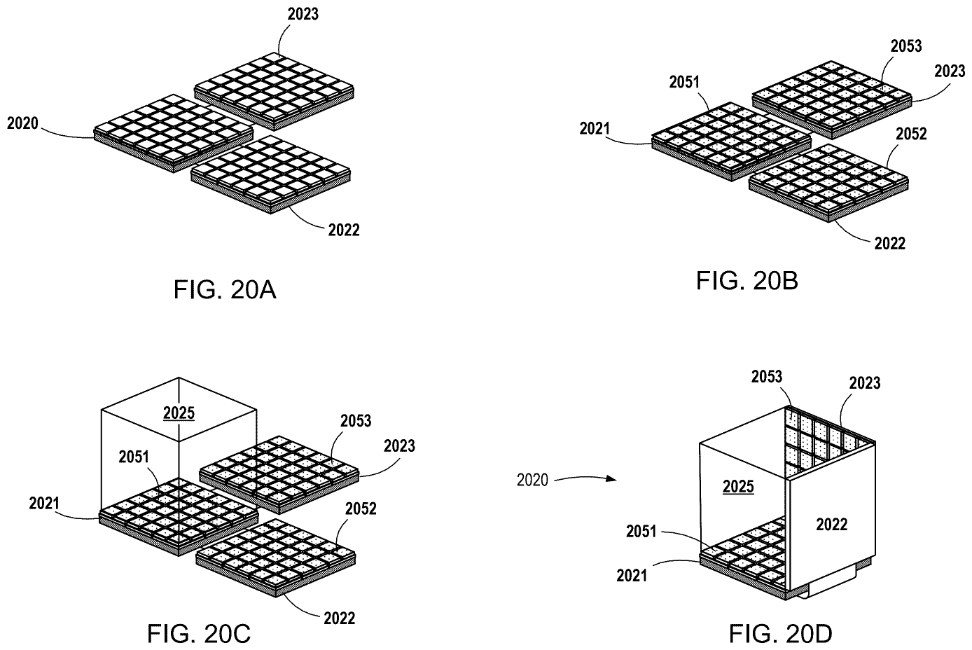

FIGS. 20A-20D illustrate spectrally selective crosstalk rejection layers applied to an SiPM flexi-circuit prior to folding against faces of a scintillation crystal, according to one embodiment.

FIG. 20A illustrates a three-array configuration of an SiPM flexi-circuit, including a first array of SiPMs 2021, a second array of SiPMs 2022, and a third array of SiPMs 2023. The first array of SiPMs 2021 is configured to be positioned adjacent a first face of a scintillation crystal (e.g., an end face “face 5”). The second array of SiPMs 2022 on the flexi-circuit can be folded against another face (e.g., “face 0”) of the scintillation crystal (e.g., orthogonal to the first face). The third array of SiPMs 2023 on the flexicircuit can be folded against another face (e.g., “face 3”) of the scintillation crystal (e.g., orthogonal to the first and second faces).

FIG. 20B illustrates that, prior to connection of the three-array SiPM flex-circuit to a scintillation crystal, spectrally selective crosstalk rejection layers 2051, 2052, and 2053 are applied to the detection faces of each of the first, second, and third arrays of SiPMs 2021, 2022, and 2023, respectively.

FIG. 20C illustrates a scintillation crystal 2025 with an end face (e.g., “face 5”) positioned on the first array of SiPMs 2021, according to one embodiment. A first spectrally selective crosstalk rejection layer 2051 is positioned between the first array of SiPMs 2021 and the end face of the scintillation crystal 2025.

FIG. 20D illustrates the scintillator detector 2020 with the flexi-circuit of SiPM arrays folded against the faces of the scintillation crystal 2025. As illustrated, the second array of SiPMs 2022 is folded against “face 0” of the scintillation crystal 2025 with a second spectrally selective crosstalk rejection layer 2052 therebetween. The third array of SiPMs 2023 is folded against “face 3” of the scintillation crystal 2025 with a third spectrally selective crosstalk rejection layer 2053 therebetween.

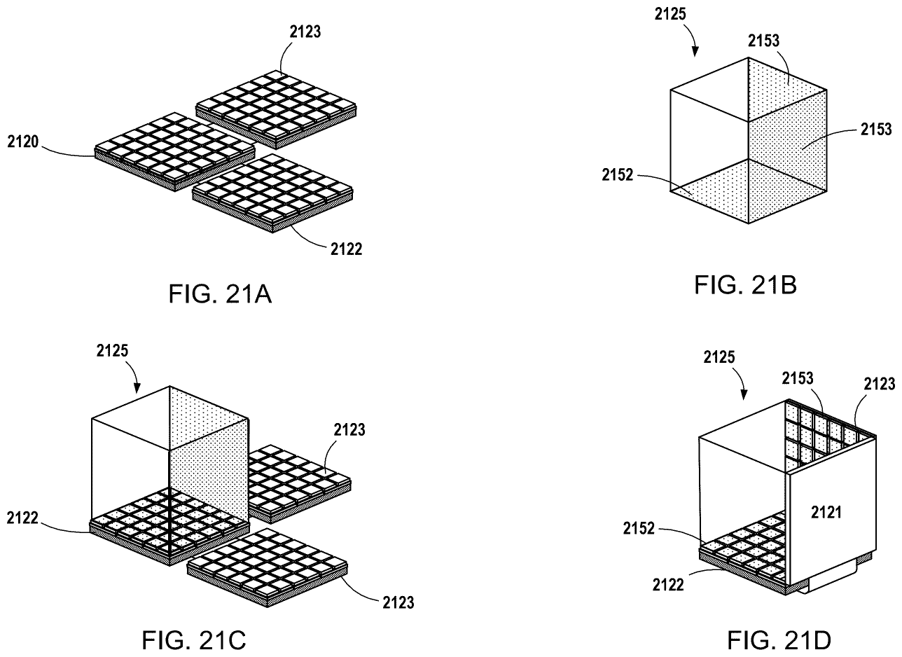

FIGS. 21A-D illustrates spectrally selective crosstalk rejection layers applied to faces of a scintillation crystal prior to applying a SiPM flexi-circuit, according to one embodiment.

FIG. 21A illustrates an SiPM flexi-circuit with three arrays of SiPMs, including a first array of SiPMs 2121, a second array of SiPMs 2122, and a third array of SiPMs 213. The first array of SiPMs 2121 is configured to be positioned adjacent a first face of a scintillation crystal (e.g., an end face “face 5”). The second array of SiPMs 2122 on the flexi-circuit can be folded against another face (e.g., “face 0”) of the scintillation crystal (e.g., orthogonal to the first face). The third array of SiPMs 2123 on the flexicircuit can be folded against another face (e.g., “face 3”) of the scintillation crystal (e.g., orthogonal to the first and second faces).

FIG. 21B illustrates a scintillation crystal 2125 with spectrally selective crosstalk rejection layers 2151, 2152, and 2153 applied to the three orthogonal faces. In the illustrated example, a first spectrally selective crosstalk rejection layer 2151 is applied to a bottom face (e.g., “face 5”), a second spectrally selective crosstalk rejection layer 2152 is applied to a second, orthogonal face (e.g., “face 0”), and a third spectrally selective crosstalk rejection layer 2153 is applied to a third, orthogonal face (e.g., “face 3”).

FIG. 21C illustrates a scintillation crystal 2125 with the end face (e.g., “face 5”) positioned on the first array of SiPMs 2121 with the first spectrally selective crosstalk rejection layer 2151 positioned between the first array of SiPMs 2121 and the end face of the scintillation crystal 2125. In the illustrated example, the second array of SiPMs 2122 and the third array of SiPMs 2123 are still in an unfolded state.

FIG. 21D illustrates the scintillator detector 2120 with the flexi-circuit of SiPM arrays folded against the faces of the scintillation crystal 2125. As illustrated, the second array of SiPMs 2122 is folded against “face 0” of the scintillation crystal 2125 with a second spectrally selective crosstalk rejection layer 2152 therebetween. The third array of SiPMs 2123 is folded against “face 3” of the scintillation crystal 2125 with a third spectrally selective crosstalk rejection layer 2153 therebetween.

Many existing computing devices and infrastructures may be used in combination with the presently described systems and methods. Some of the infrastructure that can be used with embodiments disclosed herein is already available, such as processors, microprocessors, microcontrollers, computer programming tools and techniques, digital storage media, image processor devices, imaging processing techniques, and communication links. The systems, subsystems, modules, and components may be implemented as hardware, firmware, and/or software, as understood by those of skill in the art and in the context of the associated description thereof. Various systems, subsystems, modules, and components are described in terms of the function(s) they perform because such a wide variety of possible implementations exist. For example, it is appreciated that many existing programming languages, hardware devices, frequency bands, circuits, software platforms, networking infrastructures, and/or data stores may be utilized alone or in combination to implement a specific function.

The components of some of the disclosed embodiments are described and illustrated in the figures herein. Many portions thereof could be arranged and designed in a wide variety of different configurations. Furthermore, the features, structures, and operations associated with one embodiment may be applied to or combined with the features, structures, or operations described in conjunction with another embodiment. In many instances, well-known structures, materials, or operations are not shown or described in detail to avoid obscuring aspects of this disclosure. Many of the illustrations are provided in a block diagram format to illustrate a general configuration and may not be drawn to scale. The right to add any described embodiment or feature to any one of the figures and/or as a new figure is explicitly reserved.

This disclosure has been made with reference to various exemplary embodiments, including the best mode. However, those skilled in the art will recognize that changes and modifications may be made to the exemplary embodiments without departing from the scope of the present disclosure. While the principles of this disclosure have been shown in various embodiments, many modifications of structure, arrangements, proportions, elements, materials, and components may be adapted for a specific environment and/or operating requirements without departing from the principles and scope of this disclosure. These and other changes or modifications are intended to be included within the scope of the present disclosure.

This disclosure is to be regarded in an illustrative rather than a restrictive sense. Likewise, benefits, other advantages, and solutions to problems have been described above with respect to various embodiments. However, benefits, advantages, solutions to problems, and any element(s) that may cause any benefit, advantage, or solution to occur or become more pronounced are not to be construed as a critical, required, or essential feature or element. This disclosure includes and encompasses all combinations of the described embodiments and claims that are not mutually exclusive from one another.

1. A gamma radiation scintillator detector, comprising:
a scintillation crystal to receive gamma radiation and produce scintillation photons;
a first silicon photomultiplier (SiPM) optically coupled to a first face of the scintillation crystal to generate an electric signal in response to detected photons; and
a spectrally selective crosstalk rejector positioned within an optical detection path of the first SiPM, the spectrally selective crosstalk rejector being substantially transparent to the scintillation photons and configured to absorb external crosstalk photons emitted by an avalanching single-photon avalanche diode (SPAD) that is external to the first SiPM.

2. The detector of claim 1, wherein the spectrally selective crosstalk rejector is configured to absorb external crosstalk photons with wavelengths between approximately 550 and 900 nanometers and be substantially transparent to scintillation photons with wavelengths between approximately 350 and 500 nanometers.

3-6. (canceled)

7. The detector of claim 1, wherein the spectrally selective crosstalk rejector comprises an embedded material in a protective cover of the first SiPM.

8-11. (canceled)

12. The detector of claim 1, wherein the spectrally selective crosstalk rejector comprises a polymer matrix with one or more dyes having peak absorption between 550 and 900 nanometers.

13. The detector of claim 12, wherein the polymer matrix comprises a polymethine dye with a peak absorption between 550 and 900 nanometers.

14. The detector of claim 12, wherein the polymer matrix comprises at least two dyes having different spectral absorption ranges, such that a cumulative spectral absorption range of the polymer matrix exceeds that of either dye alone.

15-17. (canceled)

18. The detector of claim 1, wherein the scintillation crystal comprises a monolithic crystal.

19. The detector of claim 1, further comprising a second SiPM optically coupled to a second face of the scintillation crystal to generate an electrical signal in response to detected photons.

20. The detector of claim 19, wherein the first face and the second face are orthogonal to one another, such that detection faces of the first SiPM and the second SiPM are orthogonal to one another.

21. The detector of claim 19, wherein the spectrally selective crosstalk rejector is positioned within the optical detection path of both the first SiPM and the second SiPM.

22. (canceled)

23. The detector of claim 19, further comprising a second spectrally selective crosstalk rejector positioned within the optical detection path of the second SiPM.

24. The detector of claim 1, wherein the spectrally selective crosstalk rejector comprises a dichroic dielectric filter.

25. (canceled)

26. (canceled)

27. The detector of claim 1, wherein the first SiPM includes trench isolation around microcells configured to suppress internal optical crosstalk.

28. (canceled)

29. The detector of claim 1, wherein the spectrally selective crosstalk rejector comprises an embedded material in a protective coating of the first SiPM.

30. (canceled)

31. The detector of claim 1, wherein the spectrally selective crosstalk rejector comprises a pigment.

32-34. (canceled)

35. The detector of claim 1, wherein the spectrally selective crosstalk rejector comprises a stack of multiple absorption layers, including at least a first absorption layer tuned to absorb a first range of optical wavelengths and a second absorption layer tuned to absorb a second range of optical wavelengths.

36. The detector of claim 1, wherein the spectrally selective crosstalk rejector is applied using a photoresist-based lithographic process.

37. (canceled)

38. The detector of claim 1, further comprising second and third SiPMs optically coupled to second and third faces of the scintillation crystal, respectively, wherein the first, second, and third faces of the scintillation crystal are mutually orthogonal, and further comprising second and third spectrally selective crosstalk rejectors positioned within optical detection paths of the second and third SiPMs, respectively, such that each of the first, second, and third faces of the scintillation crystal has a corresponding spectrally selective crosstalk rejector positioned within an optical detection path of a respective SiPM.

39-43. (canceled)

44. A gamma radiation detector module, comprising a plurality of scintillator detectors, where each scintillator detector comprises:
a scintillation crystal to receive gamma radiation and produce scintillation photons;
silicon photomultiplier (SiPM) arrays disposed on at least two different faces of the scintillation crystal; and
a spectrally selective crosstalk rejection layer positioned between each SiPM and the scintillation crystal, wherein scintillation photons generated within the scintillation crystal are detected by a SiPM after a single pass through a crosstalk-rejection layer, wherein external crosstalk photons emitted by an avalanching single-photon avalanche diode (SPAD) of one of the SIPMs on a first face of the scintillation crystal and detected by another of the SiPMs on a second face of the scintillation crystal undergo at least two passes through crosstalk-rejection layers.

45. The detector module of claim 44, wherein the scintillation crystal comprises a monolithic scintillator block.

46. The detector module of claim 44, wherein the SiPM arrays are disposed on three mutually orthogonal faces of the scintillation crystal, each face having a respective spectrally selective crosstalk rejection layer.

47. The detector module of claim 44, wherein each spectrally selective crosstalk rejection layer comprises a conformal layer.

48. The detector module of claim 44, wherein each spectrally selective crosstalk rejection layer is a dye-loaded polymer film formed from solution with a thickness of less than 10 micrometers.

49. The detector module of claim 44, wherein each spectrally selective crosstalk rejection layer comprises a conformal film disposed on a respective face of the scintillation crystal at which a corresponding SiPM is disposed.

50. The detector module of claim 44, wherein each spectrally selective crosstalk rejection layer comprises a dye-loaded polymer matrix that is substantially non-absorbing at optical wavelengths between 350 and 500 nanometers and absorbing at optical wavelengths between 550 and 900 nanometers.

51-96. (canceled)

97. A radiation detector module, comprising:
a scintillation crystal with a refractive index, nₛ; and
at least one silicon photomultiplier (SiPM) array having a pixel pitch, P, that is optically coupled to a face of the scintillation crystal via an interposed optical stack having a total thickness, d, wherein the optical stack includes a spectrally selective crosstalk rejection layer;
wherein the thickness, d, of the interposed optical stack satisfies 2d*tan θ_{s,max}≤αP, where
$${θ}_{s , max} = arcsin ( \frac{{η}_{i}}{{n}_{s}} ) ,$$
where ηᵢ is a smallest refractive index of a material in the interposed optical stack between the face of the scintillation crystal and the SiPM, and where 0<α≤0.5, such that interface-induced broadening is constrained to no more than a fraction α of the pixel pitch, P.

98. The detector module of claim 97, wherein the scintillator is monolithic.

99. The detector module of claim 97, wherein α≤0.33.

100. The detector module of claim 97, wherein the spectrally selective crosstalk rejection layer is an absorptive, conformal polymer film having a dry thickness less than 300 micrometers.

101. (canceled)

102. The detector module of claim 97, wherein the spectrally selective crosstalk rejection layer comprises a dye-loaded polymer matrix that is substantially non-absorbing at optical wavelengths between 350 and 500 nanometers and absorbing at optical wavelengths between 550 and 900 nanometers.

103. The detector module of claim 97, wherein SiPM arrays are disposed on at least two mutually orthogonal faces of the scintillation crystal, each face having a respective interposed optical stack that includes a spectrally selective crosstalk rejection layer.

104. The detector module of claim 97, wherein each SiPM includes trench isolation around microcells configured to suppress internal optical crosstalk.
